(19) United States
(12) Patent Application Publication
(10) Pub. No.: US 2025/0352805 A1
(43) Pub. Date: Nov. 20, 2025

(54) Methods and Systems for Customizing Blood Pressure Reduction Stimulation Therapy

(71) Applicant: BackBeat Medical, LLC, New Hope, PA (US)

(72) Inventors: Yuval Mika, Closter, NJ (US); Ziv Belsky, Old Tappan, NJ (US); Ricardo Aviv, Vienna (AT); Jürgen Martin Georg Bernhard, Gablitz (AT)

(21) Appl. No.: 18/866,115

(22) PCT Filed: May 24, 2023
(86) PCT No.: PCT/US2023/067392

§ 371 Date: Nov. 15, 2024

Related U.S. Application Data
(60) Provisional application No. 63/365,299, filed on May 25, 2022.

(51) Int. Cl. A61N 1/365; A61N 1/372; G16H 20/40; G16H 40/63

(52) U.S. Cl. CPC A61N 1/36564; A61N 1/37235; G16H 20/40; G16H 40/63

(57) ABSTRACT

Methods and systems for customizing blood pressure reduction stimulation therapy for a patient are disclosed, which according to an embodiment, may involve establishing baseline blood pressure parameters of a patient; applying predefined stimulation patterns and determining if any predefined stimulation patterns meet designated acceptance criteria; if no predefined stimulation patterns meet the designated acceptance criteria, calculating and applying new stimulation patterns and determining if any new stimulation patterns meet the designated acceptance criteria; determining, from among the applied predefined stimulation patterns and the applied new stimulation patterns, a stimulation pattern most suitable for the patient; and applying the most suitable stimulation pattern to the patient.

This application claims the benefit of U.S. Provisional Application No. 63/365,299, filed May 25, 2022, which is herein incorporated by reference in its entirety.

### BACKGROUND

### Field

The present embodiments relate to the field of treating hypertension, and more particularly, to methods and systems for customizing blood pressure reduction stimulation therapy for a particular patient physiology.

### Background

Methods and systems for cardiac stimulation have been developed to reduce blood pressure in patients. There remains a need to customize such stimulation therapies according to the needs of a particular patient.

### SUMMARY

Methods and systems for customizing and optimizing blood pressure reduction stimulation therapy are disclosed.

One aspect may customize and optimize therapies in the context of systems and methods for setting a stimulation pattern for continuously reducing blood pressure in a patient by reduced filling and modulating the baroreflex that responds to the reduction in pressure, such as those described in U.S. Pat. No. 10,967,188, issued Apr. 6, 2021, which is herein incorporated by reference in its entirety. In particular, embodiments may improve blood pressure reduction stimulation as therapy for hypertension by implementing a setup process that may provide specific customized and optimized stimulation parameters based on the different physiology of different patients. Embodiments may provide methods and systems for selecting an optimal set of stimulation parameters specific to a patient's particular physiology.

An embodiment may provide a method for customizing blood pressure reduction stimulation therapy for a patient. The method may comprise establishing baseline blood pressure parameters of a patient; applying predefined stimulation patterns and determining if any predefined stimulation patterns meet designated acceptance criteria; if no predefined stimulation patterns meet the designated acceptance criteria, calculating and applying new stimulation patterns and determining if any new stimulation patterns meet the designated acceptance criteria; determining, from among the applied predefined stimulation patterns and the applied new stimulation patterns, a stimulation pattern most suitable for the patient; and applying the most suitable stimulation pattern to the patient.

In an aspect, in determining if any predefined stimulation patterns meet the designated acceptance criteria and in determining if any new stimulation patterns meet the designated acceptance criteria, meeting the designated acceptance criteria may comprise calculating one or more parameters comprising an average systolic measurement value, a slope, an individual error, and/or an average error, and comparing each calculated parameter of the one or more calculated parameters to an allowed range of values for the each calculated parameter.

In another aspect, in determining if any predefined stimulation patterns meet the designated acceptance criteria and in determining if any new stimulation patterns meet the designated acceptance criteria, meeting the designated acceptance criteria may further comprise calculating a weighted score for a stimulation pattern by multiplying each of the one or more parameters (e.g., an average systolic measurement value, a slope, an individual error, and/or an average error) by a predetermined parameter weight factor, then comparing the calculated weighted score of the stimulation pattern to weighted scores of other stimulation patterns and/or to a predetermined minimal required weighted score. The predetermined parameter weight factors may reflect the relative importance of the characteristics of the therapy for different patients. In one embodiment, a lower parameter weight score may be assigned to the individual or average error in a patient known to have high blood pressure variability. In another embodiment, a lower parameter weight score may be assigned to the average systolic measurement in a patient with a less severe form of hypertension, compared to another patient with a more severe form of hypertension, which may be assigned a higher parameter weight score for the average systolic measurement.

In another aspect, the slope, the individual error, and the average error may be calculated from a linear approximation.

In another aspect, the average systolic measurement value may be calculated using only a short AV delay sub-list of systolic blood pressure values.

In another aspect, the baseline blood pressure parameters may include at least one of an average systolic measurement value, a slope of approximation, an individual error, or an average error.

In another aspect, establishing baseline blood pressure parameters of the patient may comprise measuring a pre-stimulation baseline blood pressure before applying a first stimulation pattern and during a period of no activation, applying the first stimulation pattern, measuring a first post-stimulation baseline blood pressure after applying the first stimulation pattern and during a period of no activation, and determining a first baseline blood pressure of the patient based on the measured pre-stimulation baseline blood pressure and the measured first post-stimulation baseline blood pressure. The first baseline blood pressure may be used to determine a first blood pressure effect of the first stimulation pattern, and the first blood pressure effect may be used to determine whether the first stimulation pattern meets the designated acceptance criteria.

In another aspect, determining the first baseline blood pressure of the patient may comprise calculating an average of the measured pre-stimulation baseline blood pressure and the measured first post-stimulation baseline blood pressure.

In another aspect, establishing baseline blood pressure parameters of the patient may further comprise applying a second stimulation pattern, measuring a second post-stimulation baseline blood pressure after applying the second stimulation pattern and during a period of no activation, and determining a second baseline blood pressure of the patient based on the measured first post-stimulation baseline blood pressure and the measured second post-stimulation baseline blood pressure. The second baseline blood pressure may be used to determine a second blood pressure effect of the second stimulation pattern, and the second blood pressure effect may be used to determine whether the second stimulation pattern meets the designated acceptance criteria.

In another aspect, each of the predefined stimulation patterns may comprise a cyclical stimulation pattern of several beats with a shorter atrioventricular delay followed by several beats with a longer atrioventricular delay. Calculating new stimulation patterns may include identifying, from among previously applied predefined stimulation patterns, a stimulation pattern providing a best effect; determining whether the predefined stimulation pattern providing the best effect has an acceptable level of stability; if the predefined stimulation pattern providing the best effect has an acceptable level of stability, decreasing by an increment a shorter atrioventricular delay of the predefined stimulation pattern providing the best effect and determining whether a stimulation pattern having the decreased shorter atrioventricular delay has been applied; if the stimulation pattern having the decreased shorter atrioventricular delay has not been applied, designating the stimulation pattern having the decreased shorter atrioventricular delay as a new stimulation pattern to be applied to the patient; and if the stimulation pattern having the decreased shorter atrioventricular delay has been applied, reducing a number of beats having the shorter atrioventricular delay of the stimulation pattern having the decreased shorter atrioventricular delay to provide a reduced-beat decreased shorter atrioventricular delay stimulation pattern, and designating the reduced-beat decreased shorter atrioventricular delay stimulation pattern as a new stimulation pattern to be applied to the patient.

In another aspect, the method for customizing blood pressure reduction stimulation therapy for a patient may further comprise, if the predefined stimulation pattern providing the best effect does not have an acceptable level of stability, increasing by an increment a longer atrioventricular delay of the predefined stimulation pattern providing the best effect and determining whether a stimulation pattern having the increased longer atrioventricular delay has been applied; if the stimulation pattern having the increased longer atrioventricular delay has not been applied, designating the stimulation pattern having the increased longer atrioventricular delay as a new stimulation pattern to be applied to the patient; and if the stimulation pattern having the increased longer atrioventricular delay has been applied, decreasing by an increment the shorter atrioventricular delay of the stimulation pattern having the increased longer atrioventricular delay to provide a decremented shorter atrioventricular delay stimulation pattern, and designating the decremented shorter atrioventricular delay stimulation pattern as a new stimulation pattern to be applied to the patient.

In another aspect, the method for customizing blood pressure reduction stimulation therapy for a patient may further comprise limiting the applying of the predefined stimulation patterns and the applying of the new stimulation patterns to within an allocated time based on needs of the patient.

In another aspect, the method for customizing blood pressure reduction stimulation therapy for a patient may further comprise establishing a testing protocol based on the baseline blood pressure parameters of the patient.

In another aspect, the testing protocol may comprise at least one of an overall time for completing the method; particular predefined stimulation patterns for the patient; number and/or time of the predefined stimulation patterns to be applied to the patient; or number and/or time of the new stimulation patterns to be applied to the patient.

In another aspect, the designated acceptance criteria may comprise at least one of slope and error.

In another aspect, the designated acceptance criteria may comprise at least one of a blood pressure stopping condition, a limit on blood pressure reduction, or a noise limit of measured results.

In another aspect, calculating and applying new stimulation patterns may include obtaining systolic and diastolic blood pressure values before stimulation (typically, 0-300 seconds before stimulation), during stimulation, and after stimulation (typically, 0-300 seconds after stimulation).

In another aspect, calculating and applying new stimulation patterns may include calculating intra-cycle and inter-cycle parameters.

In another aspect, the method may further include repeating the method to account for changes in patient physiology, changes in drug regimen, different times of day, different patient levels of activity, and/or different patient heart rates.

Another embodiment may provide a system for customizing blood pressure reduction stimulation therapy for a patient. The system may include at least one controller, a stimulation device configured to generate control signals associated with a heart of the patient, and a control circuit configured to deliver the control signals to the heart of the patient. The at least one controller may be configured to apply predefined stimulation patterns and determine if any predefined stimulation patterns meet designated acceptance criteria, and if no predefined stimulation patterns meet the designated acceptance criteria, calculate and apply new stimulation patterns and determine if any new stimulation patterns meet the designated acceptance criteria.

In an aspect, the at least one controller may be external to the stimulation device.

In another aspect, the system may further include a blood pressure measurement device configured to provide blood pressure data to the at least one controller.

In another aspect, the at least one controller, in determining if any predefined stimulation patterns meet the designated acceptance criteria and in determining if any new stimulation patterns meet the designated acceptance criteria, may be configured to calculate one or more parameters comprising an average systolic measurement value, a slope, an individual error, and/or an average error, and compare each calculated parameter of the one or more calculated parameters to an allowed range of values for the each calculated parameter.

In another aspect, the at least one controller, in determining if any predefined stimulation patterns meet the designated acceptance criteria and in determining if any new stimulation patterns meet the designated acceptance criteria, may be configured to calculate a weighted score for a stimulation pattern by multiplying each of the one or more parameters by a predetermined parameter weight factor, and compare the calculated weighted score of the stimulation pattern to weighted scores of other stimulation patterns and/or to a predetermined minimal required weighted score.

In another aspect, the slope, the individual error, and the average error may be calculated from a linear approximation.

In another aspect, the average systolic measurement value may be calculated using only a short atrioventricular delay sub-list of systolic blood pressure values.

In another aspect, the at least one controller may be configured to establish baseline blood pressure parameters of the patient; determine, from among the applied predefined stimulation patterns and the applied new stimulation patterns, a stimulation pattern most suitable for the patient; and apply the most suitable stimulation pattern to the patient.

In another aspect, the baseline blood pressure parameters may comprise at least one of an average systolic measurement value, a slope of approximation, an individual error, or an average error.

In another aspect, the at least one controller may be configured to establish baseline blood pressure parameters of the patient by measuring a pre-stimulation baseline blood pressure before applying a first stimulation pattern and during a period of no activation, applying the first stimulation pattern, measuring a first post-stimulation baseline blood pressure after applying the first stimulation pattern and during a period of no activation, and determining a first baseline blood pressure of the patient based on the measured pre-stimulation baseline blood pressure and the measured first post-stimulation baseline blood pressure. The first baseline blood pressure may be used to determine a first blood pressure effect of the first stimulation pattern, and the first blood pressure effect may be used to determine whether the first stimulation pattern meets the designated acceptance criteria.

In another aspect, determining the first baseline blood pressure of the patient may comprise calculating an average of the measured pre-stimulation baseline blood pressure and the measured first post-stimulation baseline blood pressure.

In another aspect, the at least one controller may be further configured to establish baseline blood pressure parameters of the patient by applying a second stimulation pattern, measuring a second post-stimulation baseline blood pressure after applying the second stimulation pattern and during a period of no activation, and determining a second baseline blood pressure of the patient based on the measured first post-stimulation baseline blood pressure and the measured second post-stimulation baseline blood pressure. The second baseline blood pressure may be used to determine a second blood pressure effect of the second stimulation pattern, and the second blood pressure effect may be used to determine whether the second stimulation pattern meets the designated acceptance criteria.

In another aspect, each of the predefined stimulation patterns may comprise a cyclical stimulation pattern of several beats with a shorter atrioventricular delay followed by several beats with a longer atrioventricular delay. The at least one controller may be configured to calculate new stimulation patterns by: identifying, from among previously applied predefined stimulation patterns, a stimulation pattern providing a best effect; determining whether the predefined stimulation pattern providing the best effect has an acceptable level of stability; if the predefined stimulation pattern providing the best effect has an acceptable level of stability, decreasing by an increment a shorter atrioventricular delay of the predefined stimulation pattern providing the best effect and determining whether a stimulation pattern having the decreased shorter atrioventricular delay has been applied; if the stimulation pattern having the decreased shorter atrioventricular delay has not been applied, designating the stimulation pattern having the decreased shorter atrioventricular delay as a new stimulation pattern to be applied to the patient; and if the stimulation pattern having the decreased shorter atrioventricular delay has been applied, reducing a number of beats having the shorter atrioventricular delay of the stimulation pattern having the decreased shorter atrioventricular delay to provide a reduced-beat decreased shorter atrioventricular delay stimulation pattern, and designating the reduced-beat decreased shorter atrioventricular delay stimulation pattern as a new stimulation pattern to be applied to the patient.

In another aspect, the at least one controller may be further configured to calculate new stimulation patterns by: if the predefined stimulation pattern providing the best effect does not have an acceptable level of stability, increasing by an increment a longer atrioventricular delay of the predefined stimulation pattern providing the best effect and determining whether a stimulation pattern having the increased longer atrioventricular delay has been applied; if the stimulation pattern having the increased longer atrioventricular delay has not been applied, designating the stimulation pattern having the increased longer atrioventricular delay as a new stimulation pattern to be applied to the patient; and if the stimulation pattern having the increased longer atrioventricular delay has been applied, decreasing by an increment the shorter atrioventricular delay of the stimulation pattern having the increased longer atrioventricular delay to provide a decremented shorter atrioventricular delay stimulation pattern, and designating the decremented shorter atrioventricular delay stimulation pattern as a new stimulation pattern to be applied to the patient.

In another aspect, the at least one controller may be configured to limit the applying of the predefined stimulation patterns and the applying of the new stimulation patterns to within an allocated time based on needs of the patient.

In another aspect, the at least one controller may be configured to establish a testing protocol based on baseline blood pressure parameters of the patient.

In another aspect, the testing protocol may comprise at least one of an overall time for completing the customizing of the blood pressure reduction stimulation therapy for the patient; particular predefined stimulation patterns for the patient; number and/or time of the predefined stimulation patterns to be applied to the patient; or number and/or time of the new stimulation patterns to be applied to the patient.

In another aspect, the designated acceptance criteria may comprise at least one of slope and error.

In another aspect, the designated acceptance criteria may comprise at least one of a blood pressure stopping condition, a limit on blood pressure reduction, or a noise limit of measured results.

In another aspect, the at least one controller may be configured to calculate new stimulation patterns by obtaining systolic and diastolic blood pressure values 0-300 seconds before stimulation, during stimulation, and 0-300 seconds after stimulation.

In another aspect, the at least one controller may be configured to calculate new stimulation patterns by calculating intra-cycle and inter-cycle parameters.

Other systems, methods, features, and advantages of the present embodiments will be, or will become, apparent to one of ordinary skill in the art upon examination of the following figures and detailed description. It is intended that all such additional systems, methods, features, and advantages be included within this description and this summary, be within the scope of the embodiments, and be protected by the following claims.

### BRIEF DESCRIPTION OF THE DRAWINGS

The embodiments can be better understood with reference to the following drawings and description. The components in the figures are not necessarily to scale, emphasis instead being placed upon illustrating the principles of the embodiments. Moreover, in the figures, like reference numerals designate corresponding parts throughout the different views.

FIG. 1 is a flow chart illustrating an exemplary method for customizing blood pressure reduction stimulation therapy, according to an embodiment;

FIG. 2 is a flow chart illustrating in more detail a portion of the method of FIG. 1, relating to the establishing of a patient's baseline blood pressure parameters and testing protocol, according to an embodiment;

FIG. 3 is a flow chart illustrating in more detail a portion of the method of FIG. 1, relating to the calculation of new stimulation patterns, according to an embodiment;

FIG. 4 is a table (Table 1) illustrating an embodiment of the method for customizing blood pressure reduction stimulation therapy of FIG. 1, listing values for therapy parameters and blood pressure measurements, according to an embodiment;

FIG. 5 is a table (Table 2) illustrating an embodiment of the method for customizing blood pressure reduction stimulation therapy of FIG. 1, listing the results of the method, according to an embodiment;

FIG. 6 is a table (Table 3) illustrating an embodiment of the method for customizing blood pressure reduction stimulation therapy of FIG. 1, in which an outlier measurement is omitted, according to an embodiment;

FIG. 7 is a schematic drawing illustrating an exemplary system for customizing blood pressure reduction stimulation therapy, which may perform one or more of the methods described herein, such as the methods of FIGS. 1-3; and

FIG. 8 is a flow chart illustrating in more detail a portion of the method of FIG. 1, relating to the application of stimulation patterns and the determination of blood pressure changes and effects, according to an embodiment.

### DETAILED DESCRIPTION

Embodiments provide methods and systems for customizing and optimizing blood pressure reduction stimulation therapy. The methods and systems may set stimulation parameters that result in a desired range of reduction in blood pressure that is maintained for a long period of time.

Embodiments of the methods and systems may enable the adjustment of stimulation pattern parameters based on a sequence of several blood pressure measurements. The sequence may include several measurements taken prior to the activation of the therapy followed by a sequence of blood pressure measurements taken following the activation of the therapy with a specified set of parameters. The method may determine a reduction in pressure and a slope of the change in the pressure that can be positive, negative, or close to zero. Based on the absolute change in blood pressure and the measured slope of the change over time, the blood pressure effect may be assigned a score that may be used for determining a best set of parameters for achieving an optimal long-term effect on blood pressure for a particular patient's physiology.

Embodiments of the methods and systems may account for the natural variability between consecutive blood pressure measurements, as well as other factors that impact blood pressure measurement, such as respiration, ectopic beats, patient state of mind, distractions, etc. The methods and systems may establish a measure of variability and determine if the measurements attained a designated minimal consistency to allow the use of these measurements. If the consistency criteria are not met, embodiments may perform additional measurements to ensure consistency.

Hypertension is defined as having a systolic (maximal high) blood pressure above a certain (normal) threshold, having a diastolic (minimal low) blood pressure above a certain (normal) threshold, or both. Hypertension treatment methods are usually measured by their reduction of systolic blood pressure (SBP). The description will therefore focus on reduction of SBP, though the same method can be applied to reduction of diastolic blood pressure (DBP).

In embodiments, methods and systems for customizing and optimizing blood pressure reduction stimulation therapy may involve: establishing a patient's baseline blood pressure parameters (e.g., average SBP, slope, error, and average error); applying predefined stimulation patterns and determining if any predefined stimulation patterns meet designated acceptance criteria; if no predefined patterns meet the criteria, calculating and applying new stimulation pattern parameters and determining if any new stimulation patterns meet the designated acceptance criteria; determining the stimulation pattern (predefined or new) most suitable for the particular patient; and applying the most suitable stimulation pattern to the particular patient. This iterative approach may establish blood pressure levels (systolic and diastolic) of the patient under different possible therapies (e.g., from both predefined and calculated stimulation patterns), allowing for therapy optimization by selecting the therapy that achieved results most appropriate for the patient.

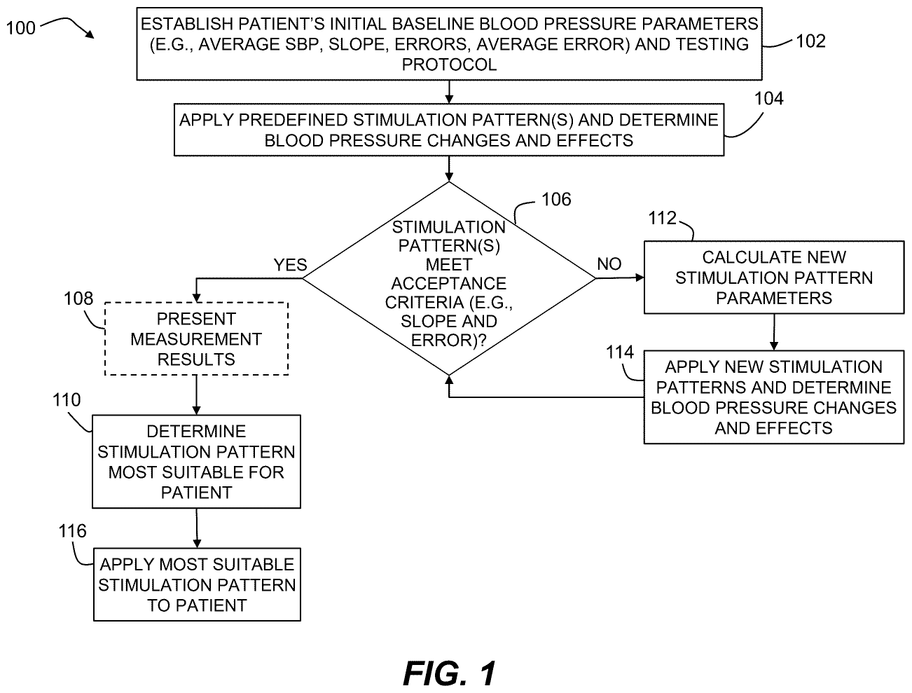

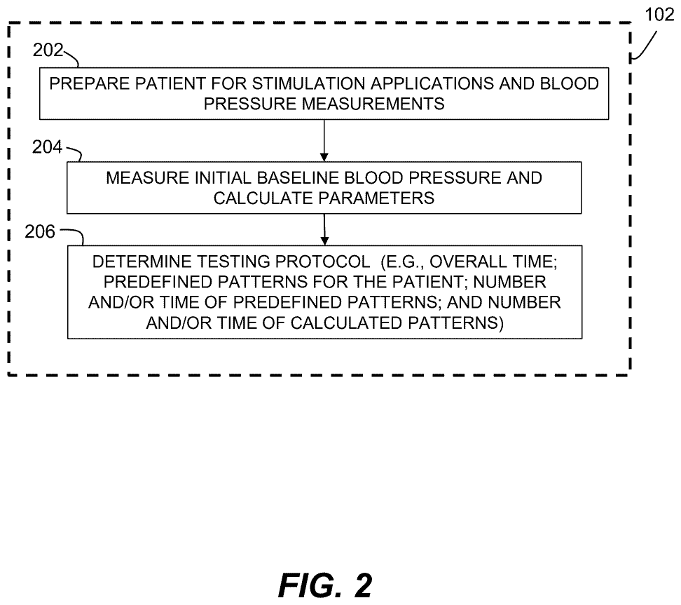

FIG. 1 illustrates a method 100 for customizing and optimizing blood pressure reduction stimulation therapy, according to an embodiment. As shown, method 100 may begin in step 102 by establishing a patient's initial baseline blood pressure parameters and a testing protocol appropriate for the patient. As shown in FIG. 2, this step 102 of establishing parameters may involve preparing the patient for stimulation applications and blood pressure measurements in step 202, measuring an initial baseline blood pressure and calculating parameters in step 204, and determining a testing protocol appropriate for the patient in step 206. Determining the protocol may include determinations of the overall time of the customization method allocated to the patient; the particular predefined stimulation patterns for the patient; the number and/or time of the predefined patterns applied to the patient; and/or the number and/or time of the calculated patterns applied to the patient.

In embodiments, measuring blood pressure may use measurement devices that can provide a reliable indication of the systolic blood pressure of the patient, as well as optionally the diastolic blood pressure, under resting conditions at a medical practitioner's office. A reliable indication means that the measurement may have a known bias from the actual systolic blood pressure, for example. Such a reliable measurement should preferably be available from the device at intervals of no more than 120 seconds apart, to provide enough information. The device may be implanted, a manual or automatic cuff-based blood measurement device, or any other device that provides suitable measurements of blood pressure.

In establishing a patient's initial baseline blood pressure parameters and testing protocol at step 102, embodiments may determine an overall time for applying the different therapies (e.g., in step 206) that is appropriate for the needs of the patient, e.g., based on the patient's initial baseline blood pressure parameters. In embodiments, the overall time may range from about 15 minutes to about 60 minutes, depending on the needs of the patient. Embodiments may measure blood pressure level (systolic and diastolic) of the patient, at either baseline level or when a certain therapy is applied, over a time of not less than about 3 minutes and no more than about 10 minutes. The upper end of the range, 10 minutes, may allow for obtaining several separate measurements of blood pressure even using slower measurement devices. The lower end of the range, 3 minutes, may be useful even if much faster devices are used, to allow for the determination of the slope (if one exists) of the change in blood pressure over time while averaging the different factors affecting blood pressure, such as breathing, temporary arrhythmia (such as premature ventricular contraction), patient cough, etc. There are different methods to obtain reliable measurements at shorter time intervals. These methods may include, without limitation: (1) continuous noninvasive blood pressure measurement using a finger cuff with an infra-red transmission plethysmograph (e.g., Finapres NOVA®); and (2) pulse transit time (PTT) or photoplethysmography (PPG) based devices (e.g., Apple Watch, Galaxy Watch).

In embodiments of step 102 and in subsequent steps evaluating possible therapies, the values of the systolic blood pressure measured, along with the times in which they were measured, may be used to calculate one or more of the following parameters:
Average Systolic Blood Pressure (SBP): the mathematical average systolic value of the measurements;
Slope: the slope of the approximation, preferably linear least squares method, of the measurements, considering both measurement time and value;
Errors (or individual errors): the calculated error (on a time-value graph) of each individual measured systolic value from the linear approximation used to calculate the slope; and
Average error: an average, preferably mathematical, of the above calculated individual errors.

In embodiments, when establishing the systolic blood pressure at baseline or as a response to a specific therapy, the number of measurements (and thus the time spent measuring) may be shortened if the obtained measurements are accurate enough. Sufficient accuracy may be determined by calculating, once enough separate measurements of systolic blood pressure are available (e.g., minimum of three), the individual errors and average error of the measurements. Sufficient accuracy may be determined in different ways, such as: comparing the average error to a predetermined threshold; comparing the average error rate of change (with additional measurements) to a predetermined threshold; and/or comparing the median error to a predefined threshold. Allowing termination of blood pressure measurements before the maximal allowed time elapsed, may either speed up the process of setting up the therapy, or allow more calculation steps and therefore possibly identification of a stimulation pattern more appropriate for the patient using the same setup time.

With the patient's initial baseline blood pressure parameters and the testing protocol established, method 100 may continue in step 104 by applying one or more predefined stimulation patterns and determining the resulting blood pressure changes and effects. Embodiments may perform measurements with at least two predefined sets of stimulation parameters. The predefined sets of stimulation parameters may be based on previously used parameters that generated desirable effects in prior patient populations. These initial, predefined sets can be adjusted based on a patient's specific physiological conditions, such as systolic blood pressure, average heart rate, diastolic blood pressure changes in blood pressure throughout the day, etc. Based on the measured effect (e.g., absolute change, slope, and error), embodiments may calculate additional sets of parameters that are more likely to generate an optimal effect based on predefined sets of rules.

There may be a tradeoff between the number of predefined stimulation patterns used as part of method 100 and the number of new, calculated stimulation patterns (discussed more below), if the overall time allocated to method 100 is kept constant. Use of more predefined stimulation patterns may allow one of them to achieve a sufficient reduction in blood pressure without the need for changing stimulation pattern parameters, applying those patterns to the patient, and measuring the effects. This could be useful, for example, for patients who require only a smaller decrease in blood pressure, which is easier to achieve. Use of fewer predefined stimulations leaves more time for new, calculated stimulations, and could be useful, for example, for patients who require a larger decrease in blood pressure. Optimally, time should be left for at least one or two iterations of changing stimulation parameters in almost any patient.

In embodiments, the overall time allocated to method 100 may be fixed (or pre-set), or set by a user. The overall time may depend on the patient. For example, a patient requiring a larger decrease in blood pressure may be allocated a longer procedure time, to allow for better therapy optimization.

Referring again to FIG. 1, method 100 may continue in step 106 by determining if any of the predefined stimulation patterns meet designated acceptance criteria for the particular patient. Acceptance criteria may be designated by user input, and may be used by method 100 to determine whether a therapy suitable for the particular patient has been achieved. Based on that determination, in step 106, method 100 either accepts a predefined stimulation pattern that met the acceptance criteria or, if no predefined stimulation pattern achieved the acceptance criteria, continues to look for a better therapy for the particular patient (proceeding to step 112, discussed below).

In embodiments, at least one of the acceptance criteria may be a stopping condition for method 100, preferably expressing a sufficient decrease in blood pressure. In embodiments, this criteria (typically expressed in mmHg reduction of blood pressure) can be the desired blood pressure change from baseline. Alternatively, this criteria can be the absolute value of systolic pressure desired for the patient, or this criteria can be the percent of reduction from the baseline pressure. This criteria, as well as other criteria, can be predefined for all patients or for patient groups (based on characteristics), or manually set by a medical practitioner for each patient.

In other embodiments, another acceptance criteria could include a limit on blood pressure reduction, e.g., to avoid a blood pressure decrease that is too large for a particular patient. Method 100 may be configured not to stop even if a large enough decrease in blood pressure was achieved, if it is deemed to be too large for the patient, so that a smaller decrease that is still large enough to be therapeutically effective, can be sought. For example, a patient with a baseline systolic blood pressure of 135 could have a stopping condition of 10 mmHg desired decrease in blood pressure, but also a “too large” decrease value of 25 mmHg, such that if a parameter set of the therapy leads to a decrease of pressure to 100 mmHg (decrease of 35 mmHg), which is not therapeutically beneficial, method 100 will not stop despite this decrease being larger than the stopping condition, because it is above the “too large” decrease value.

In other embodiments, as another example, an acceptance criteria may be based on “noise” of the measured results. Results that are “too noisy” may be, for example, an error that is too large, despite the blood pressure decrease being satisfactory. There are different possible ways to determine that an error value can be deemed “too large,” such as: the average calculated error is larger than a predefined value; the average calculated error is larger by a designated percentage (e.g., about 25%) than the largest other error value obtained in any of the other measurements performed by method 100; and/or the average calculated error is larger by a designated percentage (e.g., about 100%) from the average error value obtained in any of the other measurements performed by method 100.

Thus, as shown in FIG. 1, embodiments may involve:
1. Establishing initial baseline blood pressure parameters (e.g., average SBP, slope, errors, and average error) and a testing protocol (step 102);
2. Applying predefined stimulation pattern(s) and determining resulting blood pressure parameters (step 104);
3. Determining whether any predefined stimulation patterns meet the acceptance criteria (e.g., meet acceptable slope and error) (step 106); and
4. If not, then calculating new stimulation pattern parameters (step 112, discussed more below), and applying to the patient the new stimulation pattern parameters and determining whether any new stimulation patterns meet the acceptance criteria (e.g., based on average SBP, slope, and errors) (step 106).

In embodiments, the effect of a stimulation pattern on blood pressure may be expressed as the average SBP achieved with that pattern, minus the average SBP of the baseline blood pressure, in mmHg. In general, since any hypertension therapy aims to reduce the blood pressure, a more negative number (for the effect) is a better outcome for a stimulation pattern. However, in some cases, the reduction in blood pressure may increase the risk of hypotension and may be less desirable.

In experiments applying hypertension reduction stimulation patterns to patients, the inventors have found that in some cases, especially when the wrong pattern parameters are chosen for a particular patient, the initial reduction in blood pressure cannot be maintained over time and the pressure gradually increases over a time period of minutes. An observation period of about 3 minutes was found to be sufficient to determine if the therapeutic effect is stable over time. In embodiments, the slope calculated for the hypertension reduction stimulation pattern is an expression of this level of stability.

Based on the experiments, the inventors have also found that when using a hypertension therapy stimulation pattern that includes a cyclical stimulation pattern of several beats with a shorter AV (atrioventricular) delay followed by several beats with a longer AV delay:
1. Shortening the shorter AV delay usually causes a further reduction in systolic blood pressure;
2. Lengthening the longer AV delay usually causes a lower slope; and
3. Changing the number of beats (shorter AV delay and/or longer AV delay) mainly affects the slope.

However, there may be some limitations on these parameters' values.

In embodiments applying a cyclical stimulation pattern of several beats with a shorter AV (atrioventricular) delay followed by several beats with a longer AV delay, the shorter AV delay may be within a range of about −5 msec to about 130 msec, while the longer AV delay may within a range about 100 msec to about 240 msec.

In a practical implementation, a negative AV delay may require prediction of a naturally occurring atrial event or pacing the ventricle prior to pacing of the atrium. Therefore, in some practical implementations of the present embodiments, the minimal AV delay value allowed is 0 msec.

The shorter AV delay may be too long to effectively reduce atrial kick. In embodiments, the inventors have found that a value of the shorter AV delay of about 130 msec (when pacing both the atrium and the ventricle) is the longest to be effectively used.

A value of an AV delay that is longer than the natural AV delay means that the naturally occurring ventricular activation will start before a pacing pulse will be triggered, and may be inhibited (because of the natural activation). While the therapy will still be effective in such a case, the pacing pulse is ineffective, meaning that in embodiments, the relevant AV delay values for the method should not be longer than the natural AV delay, unless there is no natural AV delay (e.g., in the case of complete heart block). In some embodiments, the natural AV delay can be used as the longer AV delay, for example, by intentionally setting a pacing AV delay longer than the natural AV delay. These embodiments may be relevant to individuals in which A-V conduction exists, and may be less relevant to individuals with any levels of partial or complete ventricular conduction block.

In embodiments, the number of longer AV delay beats should typically be up to 5, due to the usually quick baroreflex response to an increase in blood pressure.

In embodiments, the number of shorter AV delay beats should typically be 6-18, due to the much longer time constant of the baroreflex response to a decrease in blood pressure.

In some embodiments, limits to stimulation parameters (“parameter limits”), AV delays, and beat numbers, such as using only shorter AV delays of 0 to 100 msec and only 6-18 shorter AV delay beats, are used by embodiments in calculating stimulation patterns as part of the procedure.

In some rare cases, a patient may have time constants of changes that are totally different than the ranges suggested here, in which case the present embodiments may not be as effective.

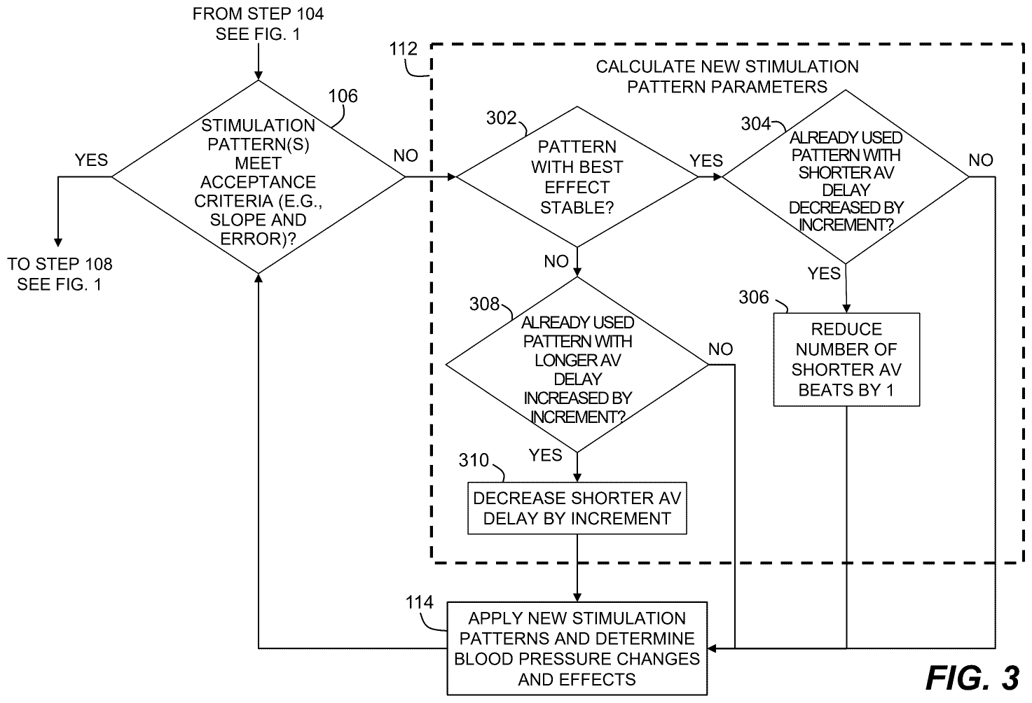

Based on experiments, the inventors found that in many cases, finding a stimulation pattern that causes a stable blood pressure response is more challenging than finding a stimulation pattern with a short-term effect that fades over time. To address that challenge, referring to FIGS. 1 and 3, embodiments of method 100 may include an iterative calculation process for calculating new stimulation pattern parameters in step 112. As shown in FIG. 3, the calculation process may begin in step 302 by determining whether the stimulation pattern with the best effect (e.g., largest decrease in blood pressure) is sufficiently stable. If so, the calculation process may proceed to step 304, which decreases the shorter AV delay used in that stimulation pattern by an increment (e.g., subtracting 10 msec off the value, but not exceeding the parameter limitations, which in this case means that the value cannot be negative) and then determines whether that decreased shorter AV delay has already been used in method 100. If a stimulation pattern with that decreased shorter AV delay has not already been used, the calculation process ends and method 100 continues to step 114 at which point the new stimulation pattern with the decreased shorter AV delay is applied to the patient and the blood pressure changes and effects are determined.

If, in step 304, it is determined that the stimulation pattern with the decreased shorter AV delay was already used, then the calculation process continues in step 306 by reducing the number of shorter AV delay beats by 1 and then in step 114 applying that new stimulation pattern and determining the blood pressure changes and effects.

Referring again to step 302, if the stimulation pattern with the best effect is not is sufficiently stable, then the calculation process may proceed to step 308, which looks for the stimulation pattern that is most stable (e.g., lowest slope) by increasing the longer AV delay used in that pattern by an increment (e.g., adding 10 msec to the value, but not exceeding the parameter limitations) and then determining whether that increased longer AV delay has already been used in method 100. If a stimulation pattern with that increased longer AV delay has not already been used, the calculation process ends and method 100 continues to step 114 at which point the new stimulation pattern with the increased longer AV delay is applied to the patient and the blood pressure changes and effects are determined.

If, in step 308, it is determined that the stimulation pattern with the increased longer AV delay was already used, then the calculation process continues in step 310 by decreasing the shorter AV delay by an increment (e.g., subtracting 10 msec off the value, but not exceeding the parameter limitations) and then in step 114 applying that new stimulation pattern and determining the blood pressure changes and effects.

As shown in FIGS. 1 and 3, after applying a new stimulation pattern at step 114, method 100 may continue in step 106 by determining whether the new stimulation pattern meets the acceptance criteria. If the new stimulation pattern does not meet the acceptance criteria, method 100 returns to the calculation process 112 to determine another new stimulation pattern.

Regarding step 106, in determining if any predefined stimulation patterns meet the designated acceptance criteria and in determining if any new stimulation patterns meet the designated acceptance criteria, meeting the designated acceptance criteria may comprise calculating one or more parameters comprising an average systolic measurement value, a slope, an individual error, and/or an average error, and comparing each calculated parameter of the one or more calculated parameters to an allowed range of values for the each calculated parameter.

In further embodiments, for step 106, in determining if any

predefined stimulation patterns meet the designated acceptance criteria and in determining if any new stimulation patterns meet the designated acceptance criteria, meeting the designated acceptance criteria may further comprise calculating a weighted score for a stimulation pattern by multiplying each of the one or more parameters (e.g., an average systolic measurement value, a slope, an individual error, and/or an average error) by a predetermined parameter weight factor, then comparing the calculated weighted score of the stimulation pattern to weighted scores of other stimulation patterns and/or to a predetermined minimal required weighted score. The predetermined parameter weight factors may reflect the relative importance of the characteristics of the therapy for different patients. In one embodiment, a lower parameter weight score may be assigned to the individual or average error in a patient known to have high blood pressure variability. In another embodiment, a lower parameter weight score may be assigned to the average systolic measurement in a patient with a less severe form of hypertension, compared to another patient with a more severe form of hypertension, which may be assigned a higher parameter weight score for the average systolic measurement.

In embodiments, method 100 may be configured to cycle repeatedly through steps 106, 112, and 114 until a predetermined and/or user-determined amount of time has passed. During that time, any stimulation patterns determined to meet the acceptance criteria may be included in the subsequent steps 108 and 110. As shown in FIG. 1, method 100 may include the optional step 108 of presenting measurement results of stimulation pattern(s) that meet the acceptance criteria. The present measurement results could include predefined stimulation patterns from step 104 and/or stimulation patterns resulting from the calculation process in step 112. Embodiments may present measurement results that only meet the acceptance criteria, while other embodiments may present all measurement results, including those that met the criteria and those that did not meet the criteria.

In embodiments, the measurement results presented at step 108 may include a list of the different stimulation patterns used, each with a set of calculated parameters, such as one or more of the following:
1. Effect—average SBP achieved with that stimulation pattern, minus the average SBP of the baseline blood pressure, which can be expressed in mmHg, since most medical practitioners are very familiar with blood pressure differences and know how to interpret them.
2. Stability—the slope calculated for that stimulation pattern. Since medical practitioners are usually not familiar with the concept of linear approximations, the results may be mapped into a set of discrete values and/or categories. In embodiments, any negative slope may be designated “very stable,” slightly positive may be designated “stable” (e.g., slope of up to 1 mmHg per minute, generally allowing up to 5 mmHg between measurements taken during 5 minutes), and a higher slope may be designated “unstable.”
3. Fluctuations—the average error calculated for that stimulation pattern. The results may also be mapped into a set of discrete values and/or categories. In embodiments, an average square error value of below 2 may be designated “very accurate,” an average square error value of above 2 but below 4 may be designated “fairly accurate,” and an average square error value of above 4 may be designated “fairly inaccurate.”

Alternatively or optionally, an updated baseline measurement can be used to calculate the “Effect” parameter. A period of time during which no stimulation is provided to the patient may precede the stimulation delivery. Measurements of systolic blood pressure during this preceding period may be averaged and subtracted from the average SBP achieved with that stimulation pattern, instead of subtracting the average SBP of the baseline blood pressure performed in the first step 102 of method 100. Optionally or additionally, a period of time during which no stimulation is provided to the patient may follow the stimulation delivery. Measurements of systolic blood pressure during this subsequent period may be averaged and subtracted from the average SBP achieved with that stimulation pattern, instead of subtracting the average SBP of the baseline blood pressure performed in the first step 102 of method 100. Optionally, measurements from periods of time without stimulation both before and after stimulation delivery may be averaged to subtract from the average SBP achieved with the stimulation pattern to calculate the Effect parameter.

Embodiments may also include provisions, such as the above-described updated baseline measurements, for accounting for changes in a patient's blood pressure caused by a method for setting up, customizing, and optimizing a blood pressure reduction stimulation therapy, such as method 100 of FIG. 1. In experiments implemented according to method 100, the application of multiple blood-pressure-modulating stimulation patterns to a patient has led to changes in the patient's baseline blood pressure, at least for short periods of time between consecutive activations of the therapeutic stimulation patterns as part of the setup, customization, and optimization process. While those changes may account for intermittent and/or short periods of progressively lowered blood pressure, in embodiments, an opposite effect may also occur in which the patient's blood pressure increases over time. That increase could be due to a patient becoming progressively more agitated or impatient with the setup process and the blood pressure measurements taken in the process. However, regardless of the reason, experiments have demonstrated that the change over time in the underlying patient “baseline” blood pressure may be negative, positive, or none at all (meaning the blood pressure remains about the same).

To account for this possible migration, or draft, in baseline blood pressure over time, embodiments may determine a blood pressure during a pause before and/or after activation of therapy (e.g., before and/or after application of a stimulation pattern) and when no activation is present. Determining the blood pressure may involve one measurement, or to increase precision, may involve multiple measurements. In embodiments, two measurements are taken and the average of the two measurements is used. If more than two measurements are used, then later measurements may be weighted more in the average, since any remaining effect from the last activation slowly decreases over time. Thus, in embodiments, blood pressure changes and effects may be determined each time based on the most recent, and therefore accurate, baseline blood pressure value.

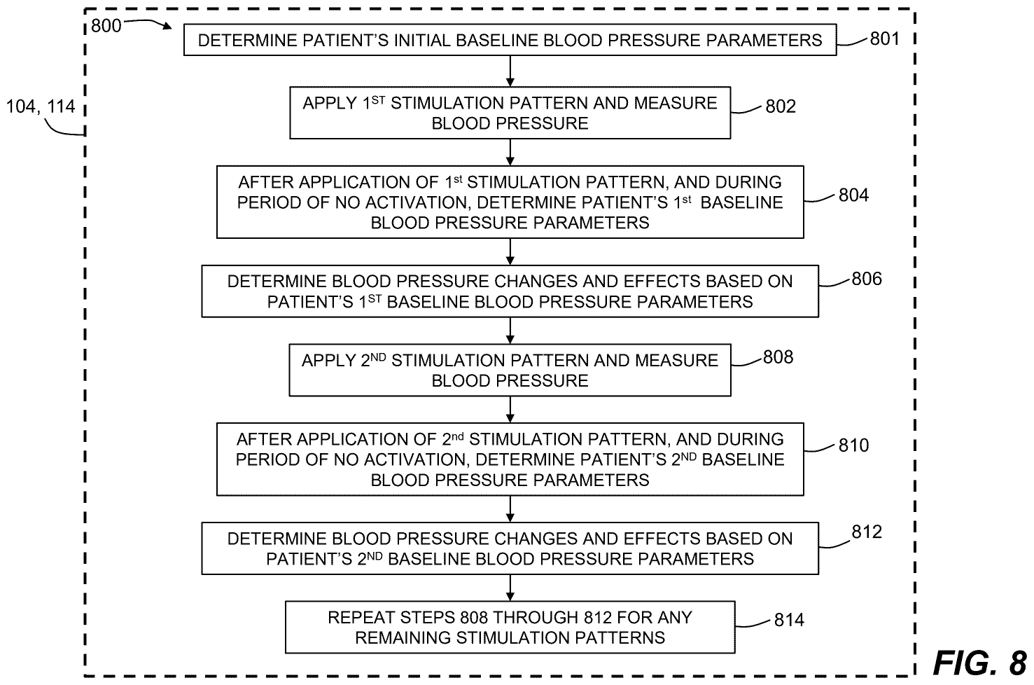

FIG. 8 illustrates an embodiment of a method 800 for accounting for changes in a patient's blood pressure in applying stimulation patterns and determining blood pressure changes and effects, which may be performed as part of steps 104 and 114 of the method 100 of FIG. 1. As shown in FIG. 8, method 800 may begin in step 801 by determining a patient's initial baseline blood pressure parameters during a period of no activation and before a first stimulation pattern is applied. Referring to FIGS. 1 and 8, the pre-stimulation determination of step 801 may occur in step 102 in preparation for the first predefined stimulation pattern applied in step 104, or may occur in steps 112 or 114 in preparation for the first stimulation pattern applied in step 114.

As shown in FIG. 8, method 800 may continue in step 802 by applying a first stimulation pattern and measuring the blood pressure during the application of the first stimulation pattern. Referring to FIG. 1, this first stimulation pattern may be the first predefined stimulation pattern of the initial predefined stimulation patterns of step 104 or the first stimulation pattern of the newly calculated stimulation patterns of step 114.

After applying the first stimulation pattern, method 800 may continue in step 804 by determining the patient's first baseline blood pressure parameters during a period of no activation. In embodiments, first baseline blood pressure parameters may be determined by measuring the patient's blood pressure after the application of the first stimulation pattern ends and when therapy is not active, and using that measured post-stimulation blood pressure and the pre-stimulation blood pressure determined in step 801, to calculate the first baseline blood pressure parameters. In embodiments, blood pressure may be measured during periods of no activation before (pre-stimulation) and after (post-stimulation) each stimulation pattern, and the baseline blood pressure may be calculated using both the pre-stimulation and post-stimulation blood pressure measurements. When multiple stimulation patterns are applied, embodiments may use the blood pressure measurements taken between consecutive stimulation patterns to calculate baseline blood pressure parameters for both the preceding stimulation pattern (as the post-stimulation blood pressure) and the subsequent stimulation pattern (as the pre-stimulation blood pressure).

Thus, embodiments may measure baseline blood pressure (i.e., blood pressure when therapy is not active) before and after every activation, including a first activation (e.g., step 802). The baseline level of a therapy activation, required to calculate the effect of that therapy activation, may be calculated using both the before (pre-stimulation) blood pressure measurements taken before the therapy was active and the after (post-stimulation) blood pressure measurements taken after the therapy was turned off. In the case of multiple therapy activations, the second period of time of post-stimulation measurements for a therapy activation is also the pre-stimulation measurement for the next therapy activation (until the last therapy activation), such that blood pressure measurements taken during each period of no activation between therapy activations may be used to calculate baseline blood pressure parameters for two therapy activations: both the one before and the one after the blood pressure measurements were taken.

Referring again to step 804, in embodiments, the first baseline blood pressure parameters may be determined by calculating an average of the blood pressure parameter measurements taken before (step 801) and after (step 804) application of the first stimulation pattern (step 802). In embodiments that average measurements, measured blood pressure parameters may be weighted such that more recent measurements have a higher weight, since any remaining effect from the last activation slowly decreases over time.

Method 800 may then continue in step 806 by determining blood pressure changes and effects caused by the first stimulation pattern, relative to the first baseline blood pressure parameters. For example, an effect may be determined by calculating the difference between the measured blood pressure of step 802 and the first baseline blood pressure. Referring to FIGS. 1 and 8, the first baseline blood pressure parameters may be determined from the baseline blood pressure parameters measured in steps 801 (pre-stimulation) and 804 (post-stimulation) for the first predefined stimulation pattern applied in step 104, or for the first newly calculated stimulation pattern in step 114.

After determining the blood pressure changes and effects for the first stimulation pattern, method 800 may continue in step 808 by applying a second stimulation pattern and measuring the blood pressure during the application of the second stimulation pattern. Referring to FIG. 1, this second stimulation pattern may be the second predefined stimulation pattern of the initial predefined stimulation patterns of step 104, or the second stimulation pattern of the newly calculated stimulation patterns of step 114.

After applying the second stimulation pattern, method 800 may continue in step 810 by determining the patient's second baseline blood pressure parameters during a period of no activation. In embodiments, second baseline blood pressure parameters may be determined by measuring the patient's blood pressure after the application of the second stimulation pattern ends and when therapy is not active, and using that measured post-stimulation blood pressure and the pre-stimulation blood pressure determined in step 804 (which was the post-stimulation blood pressure for the first stimulation pattern), to calculate the second baseline blood pressure parameters. In embodiments, the second baseline blood pressure parameters may be determined by calculating an average of the blood pressure parameter measurements taken before (step 804) and after (step 810) application of the second stimulation pattern (step 808). In embodiments that average measurements, measured blood pressure parameters may be weighted such that more recent measurements have a higher weight, since any remaining effect from the last activation slowly decreases over time.

Method 800 may then continue in step 812 by determining blood pressure changes and effects caused by the second stimulation pattern, relative to the second baseline blood pressure parameters determined in step 810. For example, an effect may be determined by calculating the difference between the measured blood pressure of step 808 and the second baseline blood pressure.

Method 800 may then continue iteratively in this same manner as represented in step 814, repeating steps 808, 810, and 812 for any remaining stimulation patterns. Thus, in embodiments, each time that blood pressure changes and effects are determined for a stimulation pattern, the most up-to-date baseline blood pressure parameters are used.

According to an embodiment of FIG. 8, a patient may have a first baseline systolic blood pressure of 142 mmHg based on measurements taken in step 801 (before application of a first stimulation pattern, or “activation”) and in step 804 (after application of the first stimulation pattern). Referring to steps 802 through 806, after applying the first stimulation pattern and measuring a systolic blood pressure of 132 mmHG during application of the first stimulation pattern, a blood pressure change of −10 mmHg may be determined based on the difference between the 142 mmHG first baseline systolic blood pressure and the 132 mmHg measured systolic blood pressure.

Then, referring to step 808, a second stimulation pattern may be applied, and a systolic blood pressure of 134 mmHg may be measured during application of that second stimulation pattern.

Then, referring to step 810, after applying the second stimulation pattern, and during a period of no activation, measurements taken before (step 804, which are also the measured post-stimulation blood pressure parameters for the first stimulation pattern) and after (step 810) application of the second stimulation pattern may show that the patient's baseline systolic blood pressure without therapy increases to 146 mmHg. That increased baseline systolic blood pressure may be designated the second baseline blood pressure parameters.

Thus, referring to step 812, the effect of the second stimulation pattern may be determined to be a blood pressure change of −12 mmHg, based on the difference between the 146 mmHg second baseline blood pressure parameters and the 134 mmHG systolic blood pressure measured during application of the second stimulation pattern. The blood pressure reduction of the second stimulation pattern is therefore more than the blood pressure reduction of the first stimulation pattern.

Then, referring to step 814, after determining the blood pressure changes and effects of the second stimulation pattern, method 800 may continue by repeating steps 808 through 812 for a third stimulation pattern, including: measuring a reduced systolic blood pressure of 130 mmHg during application of the third stimulation pattern; determining, based on measurements taken during periods of no activation before (step 810, which are also the measured post-stimulation blood pressure parameters for the second stimulation pattern) and after application of the third stimulation pattern, a third baseline blood pressure of 139 mmHG; and determining an effect of the third stimulation pattern to be a blood pressure change of −9 mmHg, based on the difference between the 139 mmHg third baseline blood pressure parameters and the 130 mmHG systolic blood pressure measured during application of the third stimulation pattern. Thus, the blood pressure reduction of the third stimulation pattern is less than both the blood pressure reductions of the first and second stimulation patterns.

Alternatively, instead of using both pre-stimulation and post-stimulation measurements to determine baseline blood pressure parameters as described above in embodiments of FIG. 8, other embodiments may determine baseline blood pressure parameters by measuring blood pressure parameters after application of a stimulation pattern, and using those measured post-stimulation blood pressure parameters in place of the pre-stimulation baseline blood pressure parameters measured before the stimulation pattern. For example, in FIG. 8, the second baseline blood pressure parameters may be determined by measuring (in step 810) blood pressure parameters after application of the second stimulation pattern, and using those measured post-stimulation blood pressure parameters in place of the measured pre-stimulation blood pressure parameters of step 804.

Another alternative embodiment may use only pre-stimulation measurements and not post-stimulation measurements, to determine baseline blood pressure parameters. For example, in FIG. 8, the second baseline blood pressure parameters may be determined based on the measured pre-stimulation blood pressure parameters of step 804 before application of the second stimulation pattern, and without consideration of any measured post-stimulation blood pressure parameters after application of the second stimulation pattern.

Another alternative embodiment may establish a patient's initial baseline blood pressure parameters during a period of no activation and before application of a first stimulation pattern, and may use those initial baseline blood pressure parameters to determine blood pressure changes and effects for two or more of the stimulation patterns. This approach may avoid measuring blood pressure parameters between the stimulation patterns, which may simplify and/or accelerate the process, and may reduce the resources (e.g., battery power) needed to complete the process. In embodiments, the initial baseline blood pressure parameters may be determined once before application of the first stimulation pattern during a period of no activation, and then used to determine blood pressure changes and effects for the first stimulation pattern and all subsequent stimulation patterns.

In embodiments, initial baseline blood pressure parameters may be entirely overlooked in favor of measurements that follow each subsequently applied stimulation pattern. However, as described above, other embodiments may account for each of the measurements, for example, by averaging them with or without weighting. In one embodiment, the “off” measurements taken after applications of the stimulation patterns and during periods of no activation, may be used to calculate the updated baseline blood pressure parameters for each stimulation pattern as the average of the “off” measurements performed just prior to the stimulation pattern and those performed after the stimulation pattern.

Referring again to FIG. 1, optionally, step 108 may include provisions for simplifying the determination of a stimulation pattern most suitable for a patient, which may be especially helpful in cases in which a healthcare professional is determining the most suitable stimulation pattern. In embodiments, in step 108, a smaller set of potential choices of stimulation parameters for the therapy may be presented by:
1. Selecting the best stimulation pattern (e.g., set of stimulation parameters) to the patient during the previous steps of method 100. The best stimulation pattern may be identified in this manner: the one with the best effect among the “very stable,” or among the “stable” if there are no “very stable” patterns, or among the “unstable” if there are no “very stable” or “stable” patterns.
2. Selecting the second best stimulation pattern applied to the patient during the previous steps of method 100, by removing from the results table the best stimulation pattern (from the selection in 1) and performing the same choice process as the selection in 1.
3. Reapplying the best stimulation pattern and the second best stimulation pattern to the patient, and presenting only the results of those two stimulation patterns to the healthcare professional for determination of the therapy.

As shown in FIG. 1, with the measurement results determined (and optionally presented in step 108), method 100 continues in step 110 by determining the stimulation pattern most suitable for the particular patient. That determination may be made by a user (e.g., healthcare professional) or may be automatically completed, e.g., by an automated computer-implemented evaluation of the measurement results according to the processes and criteria discussed herein.

With the most suitable stimulation pattern determined, method 100 may then continue to a final step 116 in which the most suitable stimulation pattern is applied to the particular patient. In embodiments, the most suitable stimulation pattern may be initiated by a user (e.g., healthcare professional). In other embodiments, if the determination of step 110 is automatically completed, the application of the most suitable stimulation pattern to the patient may also be automatically completed, e.g., by an automated computer-implemented application of the stimulation pattern.

In embodiments of steps 110 and 116, if the stimulation pattern with the largest SBP reduction effect was also “very stable,” it would probably be a suitable choice for many patients, unless the reduction is so large as to cause concern regarding hypotension. That could be the case if, for example, the patient did not take all of the patient's hypertension drugs on the day on which the parameter setting procedure was carried out. If the stimulation pattern with the largest SBP reduction effect was not “very stable,” determining (e.g., either by a medical professional or by an automated computer-implemented application) stimulation pattern parameters most suitable for a patient may depend on a comparison between the largest BP reduction effect of the pattern with the best stability achieved (“very stable,” if any) and the largest BP reduction effect of the pattern with the next best stability achieved (“stable” if any pattern achieved “very stable,” “unstable” if at least one pattern achieved “stable”). In some cases, it may be preferable to apply a less stable pattern with a larger BP reduction effect. For example, for a hypertensive patient with an SBP of 153 mmHg, it may be better to apply a “stable” pattern that achieved a 23 mmHg reduction in SBP to a “very stable” pattern that achieved a reduction of only 11 mmHg.

In some embodiments, the actual slope calculated for each stimulation pattern used (rather than, or in addition to, the designation “very stable,” “stable,” or “unstable”) may be used to determine the most suitable pattern to be applied to the patient. In further embodiments, the average error calculated for each pattern may also be used to determine the applied pattern. Such determinations may be based on score-based decision mechanism. For example, each calculated parameter may get a weight function, assigning a number for each possible value of the calculated parameter. The weights of the different calculated parameter values of each pattern may be multiplied to arrive at that pattern's score, and the pattern with the highest score may be applied to the patient. Examples of weight functions include: the weight of the effect is its absolute value times three (as this may be the most important aspect, clinically); the weight of the slope is the negative of the slope (since declining BP measurements, which are desirable, generate a negative slope linear approximation line); and/or the weight of the average error is 1 divided by the error (thus favoring small errors).

Blood pressure measurements may vary for different reasons, some related to inherent inaccuracies of the different available measurement devices and some related to physiological changes that may affect blood pressure at the exact moment of measurement (e.g., respiration, cough, sneeze, cardiac arrhythmia, etc.). Embodiments may therefore include provisions for addressing measurement anomalies. For example, embodiments may include additional steps that correct for measurement outliers, when enough measurements are available (e.g., at least three). The embodiments may add a step of correcting for measurement outliers when calculating any pattern parameters (e.g., average SBP, slope, and errors) to prevent miscalculations due to such outliers. In embodiments, a measurement outlier may be identified by examining a list of errors calculated for each measurement obtained for a pattern. If one of the errors is significantly larger than any of the rest (e.g., at least twice as large as the rest), then the calculation of the parameters (e.g., average SBP, slope, and errors) for this pattern should be repeated with the measurement associated with the large error omitted from the calculation. Other methods for detecting outliers may be used to eliminate these outliers. In embodiments, eliminating outliers may lead to identification of a stimulation pattern more appropriate for a patient, especially if the process includes more steps of calculating stimulation parameters.

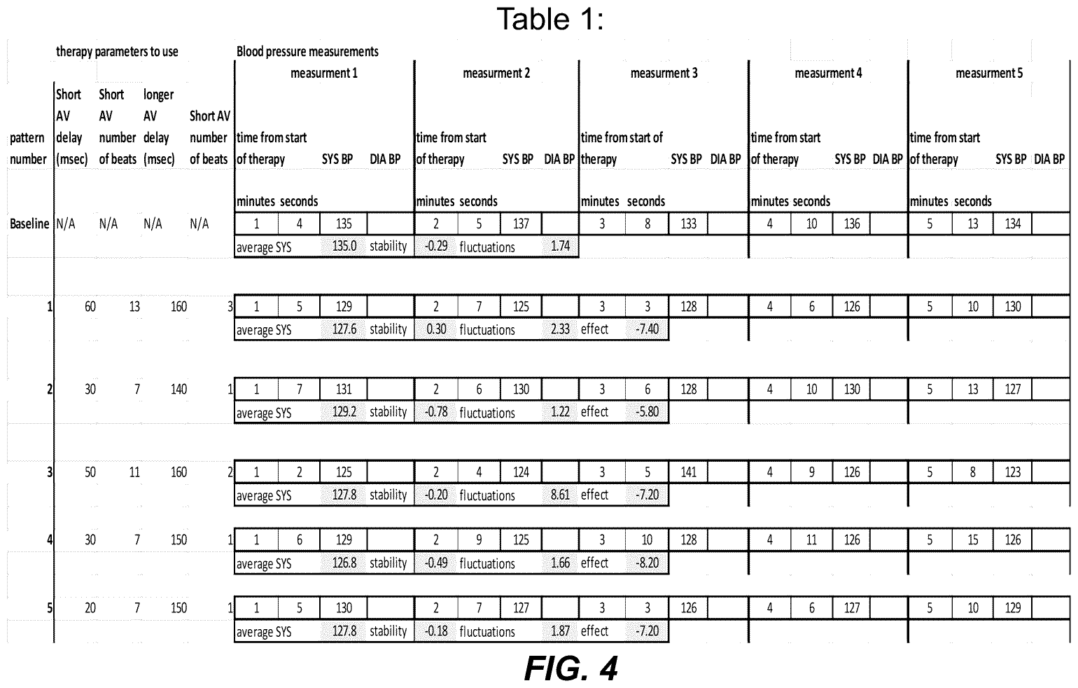

To further illustrate the method 100 of FIG. 1, FIGS. 4-6 provide measurement results of an embodiment of method 100, which used a hypertension therapy stimulation pattern that included a cyclical stimulation pattern of several beats with shorter AV delay followed by several beats with a longer AV delay.

Referring to FIG. 4, the blood pressure reduction stimulation therapy customization method was performed on a patient using a blood pressure measurement device capable of providing a value of systolic blood pressure only about every minute. The time allocated to the customization process allowed taking five measurements for each iteration of an applied stimulation pattern. A medical healthcare professional determined that a value of 10 mmHg reduction was desirable for the particular patient.

As shown in Table 1 of FIG. 4, the row titled “baseline” shows the values of systolic blood pressure values measured without therapy application, according to step 102 of method 100. For example, measurement number 1 occurred 1 minute and 4 seconds after the timepoint considered “start” of baseline, and the systolic blood pressure measured was 135 mmHg. The next measurement occurred 1 minute and 1 second later, for a total of 2 minutes and 5 seconds from the “start” of the baseline measurements, and the systolic value measured was 137 mmHg. Over the 5 measurements taken, the average systolic blood pressure level was 135 mmHg.

According to step 104 of method 100, in the embodiment, three predefined stimulation parameter sets existed to be applied to the patient. In Table 1 of FIG. 4, the therapy parameters used are shown in the appropriate row to the right of the therapy pattern number. The three predefined stimulation parameters sets are pattern numbers 1, 2, and 3. So, for example, therapy parameter set number 1 comprised applying an AV delay of 60 msec (the shorter AV delay value) for 13 consecutive beats, then applying an AV delay of 160 msec (the longer AV delay value) for 3 beats, then cycling back and applying an AV delay of 60 msec for 13 beats, and so on. Therapy parameter set number 2 comprised applying an AV delay of 30 msec (the shorter AV delay value) for 7 consecutive beats, then applying an AV delay of 140 msec (the longer AV delay value) for 1 beat, then cycling back and applying an AV delay of 30 msec for 7 beats, and so on. Therapy parameter set number 3 comprised applying an AV delay of 50 msec (the shorter AV delay value) for 11 consecutive beats, then applying an AV delay of 160 msec (the longer AV delay value) for 2 beats, then cycling back and applying an AV delay of 50 msec for 11 beats, and so on. The measurement times and systolic values measured when each of the three predefined stimulation patterns were applied to the patient also appear in Table 1 of FIG. 4.

Referring to step 106 of FIG. 1, because in this embodiment none of the first three predefined stimulation patterns achieved a reduction of 10 mmHg, method 100 proceeded to step 112 to calculate another set of parameters (another stimulation pattern). Implementing step 302 of that calculation process 112 (see FIG. 3), the stimulation pattern achieving the best effect was pattern 1 (−7.4 mmHg), but was only “stable” rather than “very stable.” Thus, moving to step 308 of the calculation process 112, the pattern that was most stable was pattern 2 (stability −0.78), so the next pattern (which was assigned to be pattern number 4) may be based on pattern 2 with an even larger value for the longer AV delay than in pattern 2. Assuming an AV delay increment of 10 msec, pattern 4 can possibly use a longer AV delay value of 150 msec instead of the 140 msec value used in pattern 2. Because a pattern applying an AV delay of 30 msec (the shorter AV delay value) for 7 consecutive values and then applying an AV delay value of 150 msec (the longer AV delay value) for 1 beat, was not yet used in this customization process, then according to the “no” branch off of step 308, that pattern was used as pattern number 4 and applied to the patient.

The measurements taken when stimulation pattern 4 was applied to the patient appear in the relevant row in Table 1 of FIG. 4. All four stimulation pattern results available were then reexamined as part of step 106 of method 100. Unfortunately, none of the four patterns achieved the desired 10 mmHg reduction in blood pressure, so another stimulation pattern was calculated to be applied. Implementing step 302 of the calculation process 112, the stimulation pattern achieving the best effect was then pattern number 4, which was also “very stable” with a stability of −0.49. Thus, according to step 304 of calculation process 112, a decrease of 10 msec of the shorter AV delay value used in pattern number 4 yielded a pattern applying an AV delay of 20 msec (the shorter AV delay value) for 7 consecutive values and then an AV delay of 150 msec (the longer AV delay value) for 1 beat, which was not yet used in the process. Thus, according to the “no” branch off of step 304, that pattern was applied as pattern number 5.

The measurements taken when stimulation pattern 5 was applied to the patient appear in the relevant row in Table 1 of FIG. 4. Though the desired effect (reduction of 10 mmHg) was not achieved yet by any of the applied parameter patterns, in this embodiment, the time allocated to the performance of the customization process ended, and the process was stopped at that point. In such situations, embodiments may consider the decision process of step 106 to be timed out and may consider measurements that have not met the acceptance criteria for the subsequent steps 108, 110, and 116.

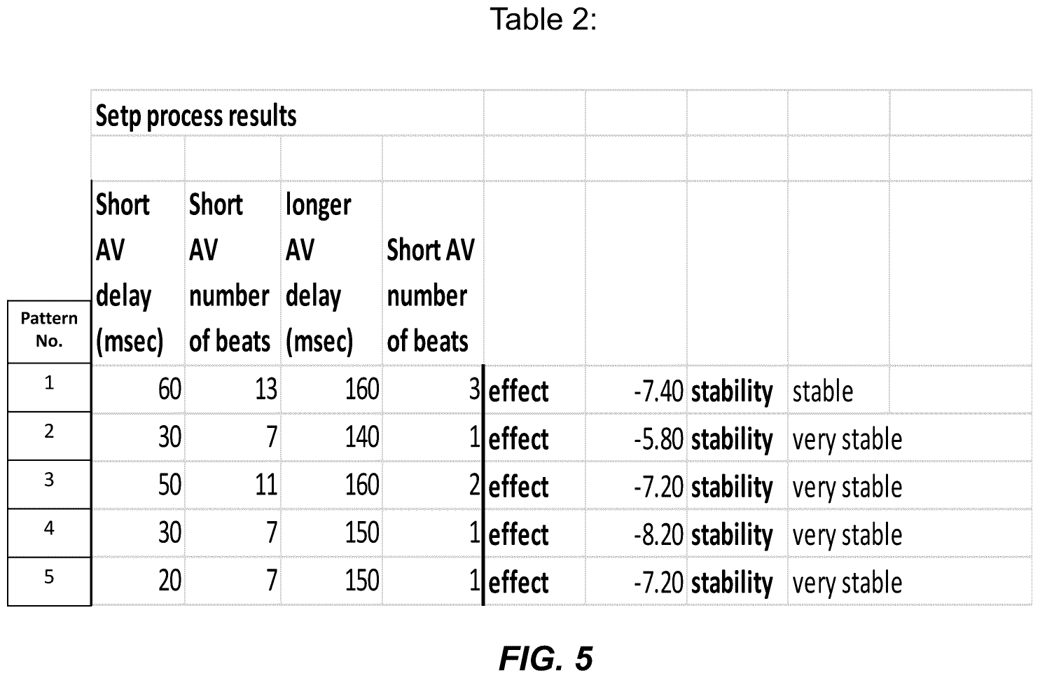

Table 2 of FIG. 5 illustrates a simplified format for presenting customization results, according to an embodiment. This simplified presentation may aid a healthcare professional in deciding that pattern 4 is best suited for this patient, according to step 110 of method 100.

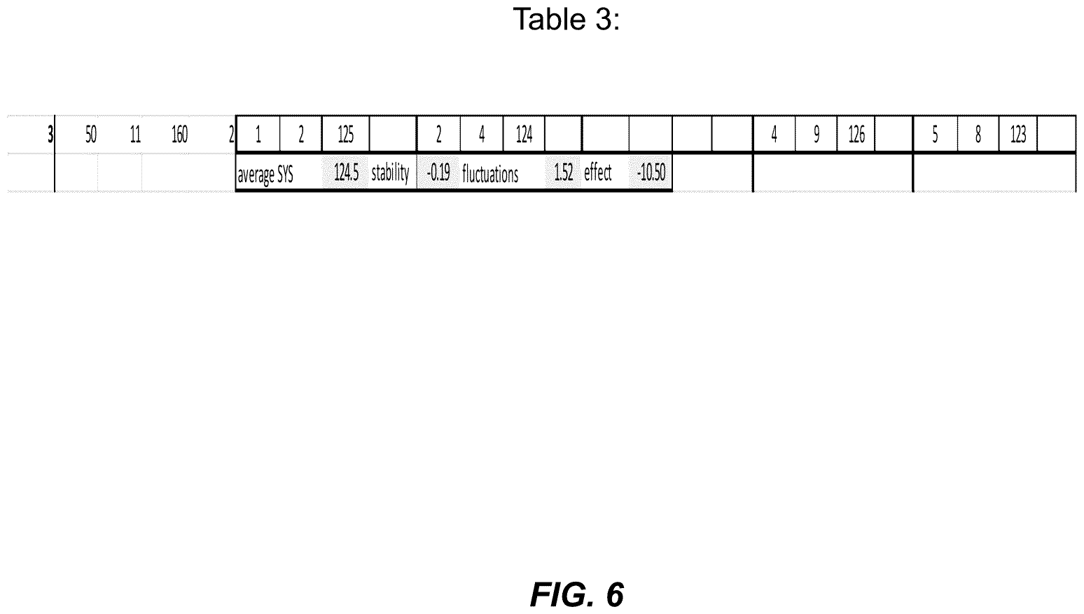

It can be noted in Table 1 of FIG. 4 that the systolic blood pressure value obtained in the third measurement (at time 3 minutes and 5 seconds) in stimulation pattern 3 was an outlier, as this value of 141 mmHg far exceeds the other values of 125, 124, 126, and 123 mmHg in pattern 3. Identifying and removing this outlier from the calculation of the effect and stability of stimulation pattern 3 (as shown in Table 3 of FIG. 6) shows an effect of −10.50 mmHg with a stability of −0.19, which is sufficient to meet the acceptable criteria. Thus, for this embodiment, using an embodiment of method 100 in which measurement outliers are removed from calculations means that the customization method 100 would have stopped after application of the first three predefined stimulation patterns, progressing through steps 104, 106 (with a “yes” condition), and 108, and without requiring calculation and application of new stimulation patterns of steps 112 and 114.

In some embodiments, beat-by-beat systolic and diastolic blood pressure data may be available, for example, when using a continuous noninvasive blood pressure measuring device such as the Caretaker Medical VitalStreams™ device manufactured by Caretaker Medical™ of Charlottesville, North Carolina. It is still possible to apply the methods as described above to calculate average systolic measurement value, slope, and error(s) using several separate blood pressure measurement values obtained during stimulation activation time, but in other embodiments systolic and diastolic blood pressure values obtained immediately before, during, and immediately after (“immediately” being 0-300 seconds, typically) can be used to perform better or additional calculations to improve the decision regarding the next set of stimulation parameters to apply, if required by the method.

It was observed by the inventors that when applying a stimulation pattern comprising short AV delay beats followed by long AV delay beats (as a repeating cyclical pattern), the short AV delay beats produced lower systolic blood pressure values than the long AV delay beats, as can be expected by the additional filling occurring during the longer AV delay period, causing higher blood pressure according to the Frank-Starling law. A chronological list of systolic blood pressure measurements obtained during the application of such a stimulation pattern may therefore be separated, based on the known number of short AV delay beats and long AV delay beats aided by expected differences in measured systolic blood pressure values in those different beats. Thus, embodiments may generate two sub-lists containing beats occurring at different time points, one being a sub-list of short AV delay systolic values and the other being a sub-list of long AV delay systolic values.

In some embodiments, the average SBP value of the stimulation may be calculated only using the short AV delay sub-list SBP values. Since, in a period of 3 to 10 minutes, there are many dozens of short AV delay beats, it is expected that such an average calculation will be more accurate than a calculation based on only a few SBP values. In some embodiments, a more accurate slope of the stimulation may also be calculated using only the short AV delay sub-list SBP values.

Intra-cycle and inter-cycle parameters may be calculated to further support parameter change decisions. One such parameter may be the difference between the last SBP value in a series of short AV delay beats and the first SBP value in the following short AV delay series of beats. Preferably, the list of all these differences calculated for all cycles of the delivered stimulation should be considered. The first SBP in the following series should be lower than the last SBP in the previous series, throughout the stimulation period, though the inventors observed in patients that it generally starts larger and decreases with time. If this parameter does not behave as expected, the number of long AV delay beats can be increased, the duration of the long AV delay can be increased, or both.

Another such parameter is the difference between the first and last SBP values in a series of short AV delay beats. Preferably, the list of all these differences calculated for all cycles of the delivered stimulation should be considered. The first SBP value should be lower than the last SBP value in the series, throughout the stimulation period, though the inventors observed in patients that the difference decreases with time. If this parameter does not behave as expected, the number of short AV delay beats can be increased, the duration of the short AV delay can be decreased, or both.

Yet another such parameter is the difference between the first and last SBP values in a series of long AV delay beats. Preferably, the list of all these differences calculated for all cycles of the delivered stimulation should be considered. The first SBP value should be higher than the last SBP value in the series, throughout the stimulation period, though the inventors observed in patients that the difference decreases with time. If this parameter does not behave as expected, the number of long AV delay beats can be increased, the duration of the long AV delay can be increased, or both.

Another such possible use comprises examining the overshoot, if it exists, when the activation is stopped. The overshoot is the difference (if it exists) between the SBP value of the first beats immediately following the termination of the stimulation, and the baseline value of the SBP. Experiments by the inventors have shown that the baseline SBP value typically returns within less than 1 minute when the stimulation activation is shorter than 10 minutes. For the purpose of calculations, the average SBP value of the first five beats after the stimulation is terminated can be compared to the average SBP of the period between 5 seconds and 300 seconds from the termination of the stimulation. In embodiments, a negative or very small overshoot value is desired, meaning the average SBP of the first five beats should be lower or equal to the average SBP of the 5-300 second period after the termination of the stimulation. If this is not the case, the number of long AV delay beats can be increased, the duration of the long AV delay can be increased, and/or the duration of the short AV delay beats can be increased.

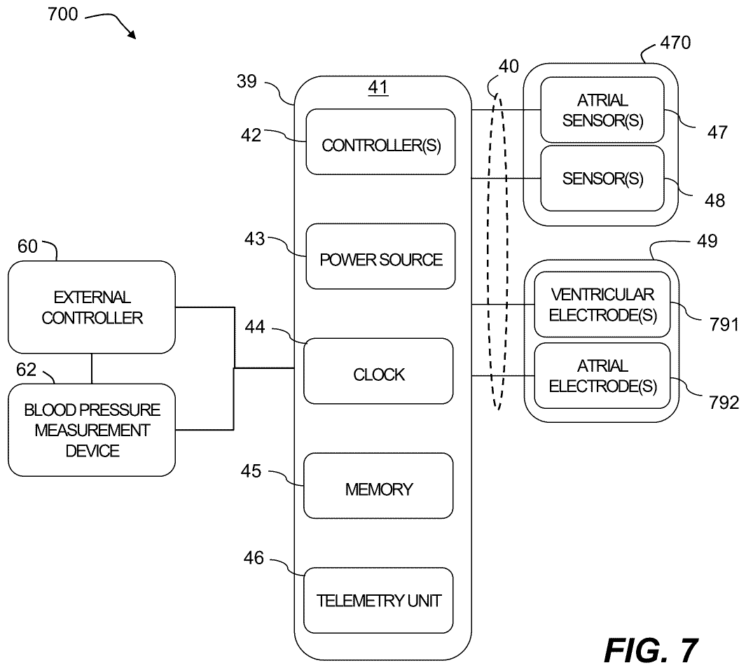

In some embodiments, an optimization of therapy may occur once, upon starting the therapy for a patient. In other embodiments, additional optimizations may occur periodically (e.g., every 12 months), to account for changes in patient physiology or drug regimen. In some embodiments, optimization may occur if the patient's systolic blood pressure exceeds a predetermined high level (e.g., 135 mmHg) over a minimal time period (e.g., three days), or in a minimal percentage of measurements over a minimal period of time (e.g., 80% of measurements in four days). Similarly, optimization may occur if a patient's systolic blood pressure decreases below a predetermined low level (e.g., 100 mmHg) over a minimal time period (e.g., 4 days) or in a minimal percentage of measurements over a minimal period of time (e.g., 70% of measurements in five days). Embodiments may measure blood pressure to determine the need for an optimization as described above, using, for example, implanted blood pressure monitors, external blood pressure monitors (e.g., a wearable watch), and/or any other suitable blood pressure measurement device (e.g., as represented by blood pressure measurement device 62 in FIG. 7, discussed below).

In some embodiments, separate optimization processes may be performed for different conditions, such as time of day, patient level of activity, and/or heart rate. The patient may be made to walk on a treadmill, for example, to increase activity level and heart rate. Different sets of parameters may result as optimal under different conditions (such as time of day or patient level of activity). In some embodiments, when different optimization results for different patient conditions are determined, different therapy parameters (as found by optimization) may then be used when different patient conditions are detected (such as time of day or activity level).

In embodiments where blood pressure data and optimizations occur repeatedly over time, the set of predefined stimulation patterns may be modified over time according to the actual optimization results obtained from the patient. In one embodiment, the optimal set of parameters obtained in the previous optimization may be used as one of the predefined stimulation patterns in the current optimization. Calculations leading to new stimulation parameters may also be impacted by patient responses to changes of parameters in previous optimization processes. In an embodiment, if a certain shortening of short AV delay led to a modest reduction of blood pressure in the past, a larger shortening of the short AV delay may be used in the next optimization process. Acceptance criteria may also be modified. In an embodiment, a higher slope or a larger error may still be deemed acceptable if a patient's responses to stimulation parameters have higher slopes or larger errors.

In embodiments where blood pressure data and optimizations occur repeatedly over time, changes in patient responses to stimulation patterns may generate relevant clinical data for a treating physician. In an embodiment, an increase (over time) of the blood pressure as a response to the same stimulation pattern may indicate a worsening patient condition. In another embodiment, increased error (blood pressure changes) to the same stimulation pattern or the average error in different stimulation patterns may indicate increased blood pressure variability.

The present embodiments may also include provisions for implementing the methods described herein, including systems and devices. The systems and devices may comprise medical systems and devices for monitoring a physiological condition of a patient and/or delivering a therapy. The devices may, for example, be implanted and/or incorporated into an external medical device, including wearable medical devices. Some implantable medical devices (IMDs), for example, may use one or more elongated electrical leads carrying stimulation electrodes, sensing electrodes, and/or other sensors. IMDs may deliver therapy to or monitor conditions of a variety of organs, nerves, muscle or tissue, such as the heart, brain, stomach, spinal cord, pelvic floor, or the like. Implantable medical leads may be configured to position electrodes or other sensors at desired locations for delivery of electrical stimulation or sensing of physiological conditions. Electrodes or sensors may, for example, be on a distal portion of a lead that is positioned subcutaneously, transvenously, or submuscularly. A proximal portion of the lead may be coupled to an implantable medical device processing unit that contains circuitry such as signal generation circuitry and/or sensing circuitry.

Embodiments may use implantable medical devices such as cardiac pacemakers or implantable cardioverter defibrillators (ICDs), which may provide therapeutic electrical stimulation to a patient's heart using signal generation circuitry of a pacemaker or ICD the delivers stimulation via electrodes of one or more implantable leads. The leads may be transvenous (e.g., advanced through vein(s) and positioned in contact with heart tissue) or non-transvenous leads implanted outside the heart (e.g., implanted epicardially, pericardially, or subcutaneously). In embodiments, electrodes may be used to sense intrinsic cardiac electrical signals for monitoring the heart rhythm and deliver electrical stimulation pulses to the heart according to desired therapies.

FIG. 7 illustrates an embodiment of a system 700 for customizing and optimizing blood pressure reduction stimulation therapy, which may include a stimulation device 41, a control circuit 40, one or more sensors 470, and one or more electrodes 49. System 700 may be constructed and have components similar to a cardiac pacemaker essentially as known in the art with some modifications as discussed herein. Optionally, one or more portions of system 700 are implantable. Optionally, system 700 comprises components that may provide additional and/or alternative electrical treatments of the heart (e.g., defibrillation). Portions of system 700 may be configured for implantation in the body of a patient essentially as is known in the art for implantable pacemakers, optionally with some modifications as discussed herein.

In embodiments, system 700 may also include an external controller 60 in communication with the stimulation device 41. External controller 60 may be configured to, for example, receive data from stimulation device 41, process the data, and control the operation of stimulation device 41. External controller 60 may also receive input from other system components and/or from users through a user interface. External controller 60 may include a display, for example, for requesting and receiving input from users, and for displaying measurement results and the status of the operation of the stimulation device 41.

In embodiments, system 700 may also include a blood pressure measurement device 62, such as those described above. Blood pressure measurement device may be in communication with the external controller 60 and/or the stimulation device 41, and may provide blood pressure measurement data.

Stimulation device 41 may include a biocompatible body 39, one or more controllers 42, a power source 43, a clock 44, a memory 45, and a telemetry unit 46. Body 39 may comprise a housing for encasing a plurality of components of the system. Controller(s) 42 may be configured to control the operation of the system, and may implement any of the embodiments and methods disclosed herein. In an embodiment, controller(s) 42 may control the delivery of stimulation pulses, the determination of predefined stimulation patterns, the calculation of new stimulation patterns, the measurement and recording of blood pressure results, the communication of measured blood pressure results for presentation of the results, the determination of stimulation pattern(s) most appropriate for a patient, and/or the application of the most suitable stimulation pattern(s) to a patient. In embodiments, controller(s) 42 may receive signals from sensors, record the signals as data in memory 45, and communicate the data using telemetry unit 46.

In some embodiments, power source 43 may include a battery. In embodiments, power source 43 may include a rechargeable battery. In some embodiments, power source 43 may include a battery that is rechargeable by induction.

In some embodiments, telemetry unit 46 may be configured to communicate with one or more other units and/or components, e.g., by radio telemetry. In embodiments, telemetry unit 46 may be configured to communicate with the external controller 60, which may be configured to, for example, program the controller(s) 42 and/or memory 45, receive data from telemetry unit 46, and/or facilitate display of data either directly or by generating signals for operation of a separate display. Telemetry unit 46 may be also be configured to communicate with a blood pressure measurement device 62.

In embodiments, blood pressure measurement device 62 and external controller 60 may be combined into a single device. In embodiments, blood pressure measurement device may be part of the stimulation device 41.

In some embodiments, system 700 may include one or more sensors 470 for sensing conditions of a patient. The sensors may be integrated into system 700, attached thereto, and/or connectable therewith. In embodiments, as shown in FIG. 7, system 700 may include one or more atrial sensors 47 for sensing the onset of atrial excitation, and/or one or more sensors 48 for providing other feedback parameters (e.g., a blood-pressure-related parameter). In some embodiments, sensor(s) 48 may comprise one or more pressure sensors, electrical sensors (e.g., ECG monitoring), flow sensors, heart rate sensors, activity sensors, and/or volume sensors. Sensor(s) 48 may include mechanical sensors and/or electronic sensors (e.g., ultrasound sensors, electrodes, and/or RF transceivers). In some embodiments, sensor(s) 48 may communicate with telemetry unit 46 of device 41 via telemetry. In some embodiments, system 700 may comprise one or more sensors for sensing one or more feedback parameters to control the application of the AV delay and/or its magnitude.

In embodiments, for example, system 700 may include one or more electrodes 49, which may apply cardiac pacing. The electrodes 49 may be integrated in system 700, attached thereto, and/or connectable therewith. In some embodiments, the electrodes 49 may include ventricular electrode(s) 791 configured to pace at least one ventricle. Additionally, system 700 may comprise one or more atrial electrode(s) 792 for pacing one or more atria.

In some embodiments, ventricular electrode(s) 791 and/or atrial electrode(s) 792 may be standard pacing electrodes. Ventricular electrode(s) 791 may be positioned relative to the heart at positions as known in the art for ventricular pacing. For example, ventricular electrode(s) may be placed in and/or near one or more of the ventricles. In some embodiments, atrial electrode(s) 792 may be placed in and/or near one or more of the atria. In some embodiments, atrial electrode(s) 792 may be attached to the one or more atria at one or more positions selected to provide early detection of atrial excitation or depolarization. For example, in some embodiments, atrial electrode(s) 792 may be attached to the right atrium near the site of the sinoatrial (SA) node.

One position of ventricular electrode(s) 791 may be such that pacing may reduce or minimize the prolongation of QRS when the heart is paced, to reduce or even minimize dyssynchrony. In some embodiments, this position is on the ventricular septum near the Bundle of His, near the Left Bundle Brunch, or in the area of Left Bundle Brunch so that is captures the Left Bundle when paced. Ventricular electrode(s) 791 may additionally or alternatively be placed on the epicardium of the heart or in coronary veins. More than one electrode can be placed on the ventricles to provide biventricular or multisite pacing, optionally to reduce dyssynchrony. Additionally or alternatively, in providing biventricular pacing, a third lead can be used to pace the left ventricle in parallel or with a fixed delay from right ventricular pacing.

Stimulation device 41 may include a pulse generator, or stimulation circuit, configured to deliver a stimulation pulse to at least one cardiac chamber. The pulse generator, or stimulation circuit, may include some or all standard capabilities of a conventional pacemaker. One or more controllers 42 may be configured to control the pulse generator, or stimulation circuit. Atrial sensor(s) 792 (and optionally other electrode sensors configured to sense other heart chambers) may be connected to system 700 via specific circuits that amplify the electrical activity of the heart and allow sampling and detection of the activation of the specific chamber. Other circuits may be configured to deliver stimulation to a specific electrode to pace the heart, creating propagating electrical activation.

In some embodiments, one or more additional sensors 48 may be placed in and/or on one or more of the atria and/or in and/or on one or more of the ventricles and/or in and/or on one or more other locations that might optionally be adjacent the heart. In embodiments, one or more sensors 48 may be placed on and/or in vena cava and/or on one or more arteries and/or within one or more cardiac chambers. These sensors 48 may measure pressure, or other indicators, such as, for example, impedance, and/or flow.

In some embodiments, one or more controllers 42 may comprise or be a microprocessor powered by power source 43. In some embodiments, stimulation device 41 may comprise a clock 44, for example, generated by a crystal. Stimulation device 41 may comprise an internal memory 45 and/or be connected to external memory. In embodiments, device may connect to an external memory via telemetry unit 46. In some embodiments, telemetry unit 46 may be configured to allow communication with external devices such as a programmer and/or one or more of sensors 48. Any and all feedback information and/or a log of device operation may be stored in internal memory 45 and/or relayed by telemetry unit 46 to an external memory unit, which may be part of, or in communication with, external controller 60.

According to further embodiments, additional systems and devices suitable for implementing the methods presented herein are described in U.S. Pat. No. 9,370,662 to Mika et al., issued Jun. 21, 2016, for example, in reference to FIGS. 9 and 14 of that patent. The entirety of U.S. Pat. No. 9,370,662 is herein incorporated by reference.

In embodiments, controllers 42 and/or controller 60 may operate in accordance with any embodiment of a method described herein.

Embodiments may implement a stimulation therapy customization and optimization method using only stimulation device 41 and without external controller 60, in which case stimulation device 41 may be preprogrammed to implement the method, such as method 100 of FIG. 1. If stimulation device 41 is an implanted medical device, such as a pacemaker-type device, then the device 41 may be configured to execute the steps of method 100 except for the optional step 108 of the presentation of measurement results, which may be omitted or may be accomplished alternatively by the device's 41 communicating of signals to an external display device, such as external controller 60, for display of the results.

Other embodiments may implement a stimulation therapy customization method using both the stimulation device 41 and the external controller 60. In such embodiments, the steps of method 100 may be shared between device 41 and external controller 60. In embodiments, stimulation device 41 may be configured to execute the steps involving application of stimulation patterns, while external controller 60 may be configured to execute the remaining steps or portions of individual steps.

In another aspect, the disclosure provides a non-transitory computer-readable medium storing software that may comprise instructions executable by one or more computers which, upon such execution, cause the one or more computers to execute any of the methods described herein.

Generally, each of device 41, external controller 60, and blood pressure measurement device 62 may include any computing device that is configured to be carried, transported, worn, or implanted in a person—and communicates wirelessly with one or more networks. In particular, an external controller 60 and/or blood pressure measurement device 62 may comprise a smartphone such as an iPhone™ or a smartphone running the Android™ operating system, or smartwatches used with those smartphones. External controller 60 and/or blood pressure measurement device 62 may broadly encompass any mobile device that includes a processor, machine readable media including electronic instructions which may be executed by the processor, and wireless networking hardware allowing the mobile device to communicate with other computing devices over a wireless network. In embodiments, external controller 60 and/or blood pressure measurement device 62 may be a mobile personal computer (e.g., laptop), a tablet computer, or even a desktop computer.

In embodiments, external controller 60 may generally be any computing device that includes a processor and machine-readable media that includes instructions that may be executed by the processor. Broadly, the processes and methods of the embodiments described in this detailed description and shown in the figures can be implemented using any kind of computing system having one or more central processing units (CPUs) and/or graphics processing units (GPUs). The processes and methods of the embodiments could also be implemented using special purpose circuitry such as an application specific integrated circuit (ASIC). The processes and methods of the embodiments may also be implemented on computing systems including read only memory (ROM) and/or random access memory (RAM), which may be connected to one or more processing units. Embodiments of computing systems and devices include, but are not limited to: servers, cellular phones, smartphones, tablet computers, notebook computers, laptop or desktop computers, all-in-one computers.

The processes and methods of the embodiments can be stored as instructions and/or data on non-transitory computer-readable media. The non-transitory computer readable medium may include any suitable computer readable medium, such as a memory, such as RAM, ROM, flash memory, or any other type of memory known in the art. In some embodiments, the non-transitory computer readable medium may include, for example, an electronic storage device, a magnetic storage device, an optical storage device, an electromagnetic storage device, a semiconductor storage device, or any suitable combination of such devices. More specific examples of the non-transitory computer readable medium may include a portable computer diskette, a floppy disk, a hard disk, magnetic disks or tapes, a read-only memory (ROM), a random access memory (RAM), a static random access memory (SRAM), a portable compact disc read-only memory (CD-ROM), an erasable programmable read-only memory (EPROM or Flash memory), electrically erasable programmable read-only memories (EEPROM), a digital versatile disk (DVD and DVD-ROM), a memory stick, other kinds of solid state drives, and any suitable combination of these exemplary media. A non-transitory computer readable medium, as used herein, is not to be construed as being transitory signals, such as radio waves or other freely propagating electromagnetic waves, electromagnetic waves propagating through a waveguide or other transmission media (e.g., light pulses passing through a fiber-optic cable), or electrical signals transmitted through a wire.

Instructions stored on the non-transitory computer readable medium for carrying out operations of the present embodiments may be instruction-set-architecture (ISA) instructions, assembler instructions, machine instructions, machine dependent instructions, microcode, firmware instructions, configuration data for integrated circuitry, state-setting data, or source code or object code written in any of one or more programming languages, including an object oriented programming language such as Smalltalk, C++, or suitable language, and procedural programming languages, such as the “C” programming language or similar programming languages.

In one or more embodiments, the functions described may be implemented in hardware, software, firmware, or any combination thereof. If implemented in software or firmware, the functions may be stored as one or more instructions or code on a computer-readable medium and executed by a hardware-based processing unit. Computer-readable media may include computer-readable storage media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor,” as used herein may refer to any of the foregoing structure or any other structure suitable for implementation of the techniques described herein. Also, the techniques could be fully implemented in one or more circuits or logic elements. The present embodiments refer to the following items:

Item 1: A system for customizing blood pressure reduction stimulation therapy for a patient, the system comprising: at least one controller; a stimulation device configured to generate control signals associated with a heart of the patient; and a control circuit configured to deliver the control signals to the heart of the patient, wherein the at least one controller is configured to: apply predefined stimulation patterns and determine if any predefined stimulation patterns meet designated acceptance criteria, and if no predefined stimulation patterns meet the designated acceptance criteria, calculate and apply new stimulation patterns and determine if any new stimulation patterns meet the designated acceptance criteria.

Item 2: In the system of item 1, the at least one controller may be external to the stimulation device.

Item 3: In the system of any of items 1 and 2, the system may further comprise a blood pressure measurement device configured to provide blood pressure data to the at least one controller.

Item 4: In the system of any of items 1-3, in determining if any predefined stimulation patterns meet the designated acceptance criteria and in determining if any new stimulation patterns meet the designated acceptance criteria, the at least one controller may be configured to: calculate one or more parameters comprising an average systolic measurement value, a slope, an individual error, and/or an average error; and compare each calculated parameter of the one or more calculated parameters to an allowed range of values for the each calculated parameter.

Item 5: In the system of any of items 1-4, in determining if any predefined stimulation patterns meet the designated acceptance criteria and in determining if any new stimulation patterns meet the designated acceptance criteria, the at least one controller may be configured to: calculate a weighted score for a stimulation pattern by multiplying each of the one or more parameters by a predetermined parameter weight factor; and compare the calculated weighted score of the stimulation pattern to weighted scores of other stimulation patterns and/or to a predetermined minimal required weighted score.

Item 6: In the system of item 4, the slope, the individual error, and the average error may be calculated from a linear approximation.

Item 7: In the system of any of items 4 and 6, the average systolic measurement value may be calculated using only a short atrioventricular delay sub-list of systolic blood pressure values.

Item 8: In the system of any of items 1-7, the at least one controller may be configured to: establish baseline blood pressure parameters of the patient; determine, from among the applied predefined stimulation patterns and the applied new stimulation patterns, a stimulation pattern most suitable for the patient; and apply the most suitable stimulation pattern to the patient.

Item 9: In the system of item 8, the baseline blood pressure parameters may comprise at least one of an average systolic measurement value, a slope of approximation, an individual error, or an average error.

Item 10: In the system of any of items 8 and 9, the at least one controller may be configured to establish baseline blood pressure parameters of the patient by: measuring a pre-stimulation baseline blood pressure before applying a first stimulation pattern and during a period of no activation; applying the first stimulation pattern; measuring a first post-stimulation baseline blood pressure after applying the first stimulation pattern and during a period of no activation; and determining a first baseline blood pressure of the patient based on the measured pre-stimulation baseline blood pressure and the measured first post-stimulation baseline blood pressure, wherein the first baseline blood pressure is used to determine a first blood pressure effect of the first stimulation pattern, and wherein the first blood pressure effect is used to determine whether the first stimulation pattern meets the designated acceptance criteria.

Item 11: In the system of item 10, determining the first baseline blood pressure of the patient may comprise calculating an average of the measured pre-stimulation baseline blood pressure and the measured first post-stimulation baseline blood pressure.

Item 12: In the system of any of items 10 and 11, the at least one controller may be further configured to establish baseline blood pressure parameters of the patient by: applying a second stimulation pattern; measuring a second post-stimulation baseline blood pressure after applying the second stimulation pattern and during a period of no activation; and determining a second baseline blood pressure of the patient based on the measured first post-stimulation baseline blood pressure and the measured second post-stimulation baseline blood pressure, wherein the second baseline blood pressure is used to determine a second blood pressure effect of the second stimulation pattern, and wherein the second blood pressure effect is used to determine whether the second stimulation pattern meets the designated acceptance criteria.

Item 13: In the system of any of items 1-12, each of the predefined stimulation patterns may comprise a cyclical stimulation pattern of several beats with a shorter atrioventricular delay followed by several beats with a longer atrioventricular delay, wherein the at least one controller may be configured to calculate new stimulation patterns by: identifying, from among previously applied predefined stimulation patterns, a stimulation pattern providing a best effect; determining whether the predefined stimulation pattern providing the best effect has an acceptable level of stability; if the predefined stimulation pattern providing the best effect has an acceptable level of stability, decreasing by an increment a shorter atrioventricular delay of the predefined stimulation pattern providing the best effect and determining whether a stimulation pattern having the decreased shorter atrioventricular delay has been applied; if the stimulation pattern having the decreased shorter atrioventricular delay has not been applied, designating the stimulation pattern having the decreased shorter atrioventricular delay as a new stimulation pattern to be applied to the patient; and if the stimulation pattern having the decreased shorter atrioventricular delay has been applied, reducing a number of beats having the shorter atrioventricular delay of the stimulation pattern having the decreased shorter atrioventricular delay to provide a reduced-beat decreased shorter atrioventricular delay stimulation pattern, and designating the reduced-beat decreased shorter atrioventricular delay stimulation pattern as a new stimulation pattern to be applied to the patient.

Item 14: In the system of item 13, the at least one controller may be further configured to calculate new stimulation patterns by: if the predefined stimulation pattern providing the best effect does not have an acceptable level of stability, increasing by an increment a longer atrioventricular delay of the predefined stimulation pattern providing the best effect and determining whether a stimulation pattern having the increased longer atrioventricular delay has been applied; if the stimulation pattern having the increased longer atrioventricular delay has not been applied, designating the stimulation pattern having the increased longer atrioventricular delay as a new stimulation pattern to be applied to the patient; and if the stimulation pattern having the increased longer atrioventricular delay has been applied, decreasing by an increment the shorter atrioventricular delay of the stimulation pattern having the increased longer atrioventricular delay to provide a decremented shorter atrioventricular delay stimulation pattern, and designating the decremented shorter atrioventricular delay stimulation pattern as a new stimulation pattern to be applied to the patient.

Item 15: In the system of any of items 1-14, the at least one controller may be configured to limit the applying of the predefined stimulation patterns and the applying of the new stimulation patterns to within an allocated time based on needs of the patient.

Item 16: In the system of any of items 1-15, the at least one controller may be configured to establish a testing protocol based on baseline blood pressure parameters of the patient.

Item 17: In the system of item 16, the testing protocol may comprise at least one of an overall time for completing the customizing of the blood pressure reduction stimulation therapy for the patient; particular predefined stimulation patterns for the patient; number and/or time of the predefined stimulation patterns to be applied to the patient; or number and/or time of the new stimulation patterns to be applied to the patient.

Item 18: In the system of any of items 1-17, the designated acceptance criteria may comprise at least one of slope and error.

Item 19: In the system of any of items 1-18, the designated acceptance criteria may comprise at least one of a blood pressure stopping condition, a limit on blood pressure reduction, or a noise limit of measured results.

Item 20: In the system of any of items 1-19, the at least one controller may be configured to calculate new stimulation patterns by obtaining systolic and diastolic blood pressure values 0-300 seconds before stimulation, during stimulation, and 0-300 seconds after stimulation.

Item 21: In the system of any of items 1-20, the at least one controller may be configured to calculate new stimulation patterns by calculating intra-cycle and inter-cycle parameters.

The foregoing disclosure has been presented for purposes of illustration and description. It is not intended to be exhaustive or to limit the embodiments to the precise forms disclosed. Many variations and modifications of the embodiments described herein will be apparent to one of ordinary skill in the art in light of the above disclosure.

While various embodiments have been described, the description is intended to be exemplary, rather than limiting and it will be apparent to those of ordinary skill in the art that many more embodiments and implementations are possible that are within the scope of the embodiments. Any feature of any embodiment may be used in combination with or substituted for any other feature or element in any other embodiment unless specifically restricted. Accordingly, the embodiments are not to be restricted except in light of the attached claims and their equivalents. Also, various modifications and changes may be made within the scope of the attached claims.

Further, in describing representative embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the present embodiments.

1. A method for customizing blood pressure reduction stimulation therapy for a patient, the method comprising:
establishing baseline blood pressure parameters of a patient;
applying predefined stimulation patterns and determining if any predefined stimulation patterns meet designated acceptance criteria;
if no predefined stimulation patterns meet the designated acceptance criteria, calculating and applying new stimulation patterns and determining if any new stimulation patterns meet the designated acceptance criteria;
determining, from among the applied predefined stimulation patterns and the applied new stimulation patterns, a stimulation pattern most suitable for the patient; and
applying the most suitable stimulation pattern to the patient.

2. The method of claim 1, wherein in determining if any predefined stimulation patterns meet the designated acceptance criteria and in determining if any new stimulation patterns meet the designated acceptance criteria, meeting the designated acceptance criteria comprises:
calculating one or more parameters comprising an average systolic measurement value, a slope, an individual error, and/or an average error; and
comparing each calculated parameter of the one or more calculated parameters to an allowed range of values for the each calculated parameter.

3. The method of claim 1, wherein in determining if any predefined stimulation patterns meet the designated acceptance criteria and in determining if any new stimulation patterns meet the designated acceptance criteria, meeting the designated acceptance criteria further comprises:
calculating a weighted score for a stimulation pattern by multiplying each of the one or more parameters by a predetermined parameter weight factor; and
comparing the calculated weighted score of the stimulation pattern to weighted scores of other stimulation patterns and/or to a predetermined minimal required weighted score.

4. The method of claim 2, wherein the slope, the individual error, and the average error are calculated from a linear approximation.

5. The method of claim 2, wherein the average systolic measurement value is calculated using only a short atrioventricular delay sub-list of systolic blood pressure values.

6. The method of claim 1, wherein the baseline blood pressure parameters comprise at least one of an average systolic measurement value, a slope of approximation, an individual error, or an average error.

7. (canceled)

8. (canceled)

9. (canceled)

10. (canceled)

11. (canceled)

12. (canceled)

13. (canceled)

14. (canceled)

15. The method of claim 1, wherein the designated acceptance criteria comprise at least one of slope and error.

16. The method of claim 1, wherein the designated acceptance criteria comprise at least one of a blood pressure stopping condition, a limit on blood pressure reduction, or a noise limit of measured results.

17. The method of claim 1, wherein calculating and applying new stimulation patterns comprises obtaining systolic and diastolic blood pressure values 0-300 seconds before stimulation, during stimulation, and 0-300 seconds after stimulation.

18. (canceled)

19. The method of claim 1, further comprising repeating the method to account for changes in patient physiology, changes in drug regimen, different times of day, different patient levels of activity, and/or different patient heart rates.

20. A system for customizing blood pressure reduction stimulation therapy for a patient, the system comprising:
at least one controller;
a stimulation device configured to generate control signals associated with a heart of the patient; and
a control circuit configured to deliver the control signals to the heart of the patient,
wherein the at least one controller is configured to:
apply predefined stimulation patterns and determine if any predefined stimulation patterns meet designated acceptance criteria, and
if no predefined stimulation patterns meet the designated acceptance criteria, calculate and apply new stimulation patterns and determine if any new stimulation patterns meet the designated acceptance criteria.

21. The system of claim 20, wherein the at least one controller is external to the stimulation device.

22. The system of claim 20, further comprising a blood pressure measurement device configured to provide blood pressure data to the at least one controller.

23. The system of claim 20, wherein in determining if any predefined stimulation patterns meet the designated acceptance criteria and in determining if any new stimulation patterns meet the designated acceptance criteria, the at least one controller is configured to:
calculate one or more parameters comprising an average systolic measurement value, a slope, an individual error, and/or an average error; and
compare each calculated parameter of the one or more calculated parameters to an allowed range of values for the each calculated parameter.

24. The system of claim 20, wherein in determining if any predefined stimulation patterns meet the designated acceptance criteria and in determining if any new stimulation patterns meet the designated acceptance criteria, the at least one controller is configured to:
calculate a weighted score for a stimulation pattern by multiplying each of the one or more parameters by a predetermined parameter weight factor; and
compare the calculated weighted score of the stimulation pattern to weighted scores of other stimulation patterns and/or to a predetermined minimal required weighted score.

25. The system of claim 23, wherein the slope, the individual error, and the average error are calculated from a linear approximation.

26. The system of claim 23, wherein the average systolic measurement value is calculated using only a short atrioventricular delay sub-list of systolic blood pressure values.

27. The system of claim 20, wherein the at least one controller is configured to:
establish baseline blood pressure parameters of the patient;
determine, from among the applied predefined stimulation patterns and the applied new stimulation patterns, a stimulation pattern most suitable for the patient; and
apply the most suitable stimulation pattern to the patient.

28. The system of claim 27, wherein the baseline blood pressure parameters comprise at least one of an average systolic measurement value, a slope of approximation, an individual error, or an average error.

29. (canceled)

30. (canceled)

31. (canceled)

32. (canceled)

33. (canceled)

34. (canceled)

35. (canceled)

36. (canceled)

37. The system of claim 20, wherein the designated acceptance criteria comprise at least one of slope and error.

38. The system of claim 20, wherein the designated acceptance criteria comprise at least one of a blood pressure stopping condition, a limit on blood pressure reduction, or a noise limit of measured results.

39. The system of claim 20, wherein the at least one controller is configured to calculate new stimulation patterns by obtaining systolic and diastolic blood pressure values 0-300 seconds before stimulation, during stimulation, and 0-300 seconds after stimulation.

40. (canceled)
